(19) United States
(12) Patent Application Publication
(10) Pub. No.: US 2025/0340562 A1
(43) Pub. Date: Nov. 6, 2025

(54) CHROMANE IMAGING LIGANDS

(71) Applicant: The General Hospital Corporation, Boston, MA (US)

(72) Inventors: Gengyang Yuan, North Reading, MA (US); Anna-Liisa Brownell, Salem, MA (US)

(21) Appl. No.: 18/573,372

(22) PCT Filed: Jun. 30, 2022
(86) PCT No.: PCT/US22/35768

§ 371 Date: Dec. 21, 2023

Related U.S. Application Data
(60) Provisional application No. 63/217,044, filed on Jun. 30, 2021.

(51) Int. Cl. C07D 491/052; A61K 51/04

(52) U.S. Cl. CPC C07D 491/052; A61K 51/0455; A61K 2123/00

(57) ABSTRACT

The present application provides radioisotope-containing compounds that are, e.g., mGluR2 modulators. Methods of imaging brain of a patient, as well as methods of diagnosing and monitoring treatment of psychiatric or neurological disorders in which mGluR2 is implicated, are also disclosed.

### CLAIM OF PRIORITY

This application claims priority U.S. Patent Application Ser. No. 63/217,044, filed on Jun. 30, 2021, the entire contents of which are hereby incorporated by reference.

### FEDERALLY SPONSORED RESEARCH OR DEVELOPMENT

This invention was made with Government support under Grant No. NS100164 and EB021708 awarded by the National Institutes of Health. The Government has certain rights in the invention.

### SEQUENCE LISTING

This application contains a Sequence Listing that has been submitted electronically as an ASCII text file named “29539-0610US1_SL_ST25.txt.” The ASCII text file, created on Jul. 26, 2024, is 7,856 bytes in size. The material in the ASCII text file is hereby incorporated by reference in its entirety.

### TECHNICAL FIELD

The current disclosure relates to advancing the chromane and isochromane negative allosteric modulators (NAMs) to suitable positron emission tomography (PET) radioligands to image metabotropic glutamate receptor 2 (mGluR2).

### BACKGROUND

There are numerous deadly diseases affecting current human population. For example, psychiatric and neurodegenerative diseases affect a significant segment of population. As one example, Parkinson's disease (“PD”), a progressive nervous system disorder that affects movement, affects more than 10 million people worldwide with an estimated total annual economic burden of more than $52 billion. In another example, schizophrenia is a long-term mental disorder of a type involving a breakdown in the relation between thought, emotion, and behavior, leading to faulty perception, inappropriate actions and feelings, withdrawal from reality and personal relationships into fantasy and delusion, and a sense of mental fragmentation. Economic burden of schizophrenia in the US exceeds $155 Billion. Currently, there is no cure for these conditions, and only therapeutic approaches that alleviate some of the symptoms are available.

### SUMMARY

The present disclosure provides, inter alia, a proof of concept study with the 5-(2-fluoro-4-([¹¹C]-methoxy)phenyl)-2,2-dimethyl-3,4-dihydro-2H-pyrano[2,3-b]pyridine-7-carboxamide, a methyl ether analogue of the chromane NAM 5-(2,4-difluorophenyl)-2,2-dimethyl-3,4-dihydro-2H-pyrano[2,3-b]pyridine-7-carboxamide, in rats and a non-human primate showed excellent brain permeability and satisfactory brain heterogeneity of the labeled in mapping the biodistribution of mGlu2 receptors in the brain. The selective binding nature of the radiolabeled compound has been confirmed by the blocking studies with MNI-137, a group II NAM, and VU60011966, a potent mGluR2 NAM. In comparison, the previously reported mGluR2 NAM radioligands were either substrates of the transporter proteins on blood-brain barrier resulting in poor brain uptake or with insufficient brain heterogeneity. In addition, the previous tracer characterizations were limited to in vitro autoradiography and imaging studies in rodents. Herein, evaluation of the PET radioligands in primate brain not only clearly outlined the radioactivity distribution in the brain but allowed the parallel blood analysis to investigate tracer metabolism and provide input function for kinetic modeling.

In one general aspect, the present disclosure provides a compound of Formula (I):

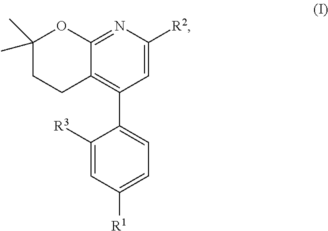

or a pharmaceutically acceptable salt thereof, wherein R¹, R², and R³ are as described herein, and one of R¹, R², and R³ comprises a radioisotope selected from ¹¹C and ¹⁸F.

In another general aspect, the present disclosure provides a pharmaceutical composition comprising a compound of Formula (I), or a pharmaceutically acceptable salt thereof, and a pharmaceutically acceptable carrier.

In yet another general aspect, the present disclosure provides a method of imaging a brain of a subject, the method comprising:
i) administering to the subject an effective amount of a compound of Formula (I), or a pharmaceutically acceptable salt thereof, or a pharmaceutical composition comprising same;
ii) waiting a time sufficient to allow the compound to accumulate in the brain to be imaged; and
iii) imaging the brain with an imaging technique.

In some embodiments, the method of imaging a brain of the subject comprises diagnosing the subject with a psychiatric or a neurological disorder associated with mGluR2.

In yet another general aspect, the present disclosure provides a method of monitoring treatment of a psychiatric or a neurological disorder associated with mGluR2 in a subject, the method comprising:
i) administering to the subject an effective amount of a compound of Formula (I), or a pharmaceutically acceptable salt thereof, or a pharmaceutical composition comprising same;
ii) waiting a time sufficient to allow the compound of Formula (I) administered in step i) to accumulate in a brain of the subject;
iii) imaging the brain of the subject with an imaging technique;
iv) administering to the subject a therapeutic agent in an effective amount to treat the psychiatric or the neurological disorder;
v) after iv), administering to the subject an effective amount of a compound of Formula (I), or a pharmaceutically acceptable salt thereof, or a pharmaceutical composition comprising same;
vi) waiting a time sufficient to allow the compound of Formula (I) administered in step v) to accumulate in the brain of the subject;
vii) imaging the brain of the subject with an imaging technique; and
viii) comparing the image of step iii) and the image of step vii).

In some embodiments, the imaging technique is selected from positron emission tomography (PET) imaging, positron emission tomography with computer tomography (PET/CT) imaging, and positron emission tomography with magnetic resonance (PET/MRI) imaging.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 Structures of the mGluR2 allosteric modulators.

FIG. 2 Characterization of compounds 12 and 13. (A) GPCR CAMP modulation result for compound 13; (B) Pgp-Glo™ assay; (C) snapshots of the docking poses for compounds 12 and 13. The key binding residues are shown in gray and the ones interacting with the ligand are labelled. The ligand atoms are rendered as carbon, nitrogen, oxygen, and fluorine in different shades. Dotted lines represent H-bonds and cyan dotted lines show π-π stacking. Pictures were rendered in PyMol 2.3.3. (D) Table 1 containing summary of the in vitro and in silico results of compounds 12 and 13.

FIG. 3 Preliminary PET imaging results of [¹¹C]13 in rat brain. (A) Summed PET images at the time interval of 1-30 min. Coronal levels show striatum (1), cingular cortex, striatum, thalamus, hypothalamus (2), cortex, hippocampus, thalamus (3), and cerebellar structures (4 and 5); (B) Representative time-activity curves of [¹¹C]13 across the regions of interest; (C) Accumulation of radioactivity during the 2-30 min window after pretreatment with VU6001966 (9) or MNI-137 (26) administered 1 min or 20 min before radioligand. The “Baseline” SUV values were the average of three baseline studies. Pictures were rendered from Prism 9.0.

FIG. 4 [¹¹C]13 analysis in arterial blood. (A) Plasma/Whole blood ratios. (B) Representative radiochromatogram of plasma samples from baseline study. (C) Individual time course of percent parent in plasma (% PP). (D) Individual metabolite-corrected SUV time courses in plasma.

FIG. 5 Characterization of [¹¹C]13 in the nonhuman primate brain. (A) 2-tissue compartment model (2T4k1v) fits in the six brain regions (left) and Logan plots (right) for [¹¹C]13 in the baseline and blocking experiments. (B) Structural MRI (MEMPRAGE) and [¹¹C]13 Logan V_{T} images for the baseline (middle) and blocking studies (bottom). (C) Logan V_{T} values bar graph obtained when using 120 min of data and t* of 30 min under baseline and blocking conditions.

FIG. 6 contains Scheme 1 showing synthesis of compounds 12, 13 and 24. Reagents and conditions: (a) THF, N₂, 80° C., 2 h; (b) RhCl(PPh₃)₃, H₂, 40 psi, rt, 2 d; (c) Pd(dppf)Cl₂, NaHCO₃, 1,4-dioxane/water, 100° C., 3 h; (d) MeMgBr (3.0 M in diethyl ether), THF, 0° C., 1 h; (e) Cs₂CO₃, DMA, 120° C., overnight; (f) Zn(CN)₂, microwave, 160° C., 30 min; (g) Na₂CO₃·1.5H₂O₂, acetone, water, rt, overnight; (h) EtOAc, H₂, Pd/C (10 wt. %), rt, overnight.

FIG. 7A contains Scheme 2 showing radiolabeling of compound 13. Compound 24 (1.6 μmol), [¹¹C]CH₃I (7.4-74 GBq), 0.5N NaOH (3.0 μL), DMF (0.35 mL), 80° C., 3 min.

FIG. 7B contains Scheme 2 showing radiolabeling strategy for compound 12 to prepare compound [¹⁸F]mG2N002 using boronic ester intermediate. See Example 1 for experimental details.

FIG. 7C contains a scheme showing radiolabeling strategy for compound 12. Reagents and conditions: (a) 21a (0.32 μmol), Pd(PPh₃)₄ (0.9 μmol), [¹¹C]HCN (3.5 GBq), DMF (0.1 mL), 160° C., 5 or 10 min; (b) Na₂CO₃·1.5H₂O₂ or 35% H₂O₂, rt, 2 min.

FIG. 8A contains images referred in the examples section as Figure S1. The figures shows initial model and all hybridized parts.

FIG. 8B contains a table referred in the examples section is Table S1. The table contains Z-Scores for the hybrid model generated on YASARA.

FIG. 9A contains an image referred to in the examples as Figure S2. The image was generated by ModFOLD based on residue accuracy prediction for the model. Various accuracy is shown.

FIG. 9B contains an images referred to in the examples as Figure S3. Verify 3D scores for the hybrid model.

FIG. 9C contains an image referred to in the examples as Figure S4. ERRAT scores for the hybrid model.

FIG. 10A contains an image referred to in the examples as Figure S5. Image shown is generated by QMEAN showing the local quality of the hybrid model. Various quality regions are shown.

FIG. 10B contains an image referred to in the examples as Figure S6. Image shows the local quality of the structure as a function of sequence number, generated by QMEAN.

FIG. 10C contains an image referred to in the examples as Figure S7. A Ramachandran plot for the hybrid model. Plot generated with the SAVES server.

FIG. 10D contains an image referred to in the examples as Figure S8. Position of the allosteric binding site for NAMs.

FIG. 11A contains images referred to in the examples as Figure S9. Purification of [¹¹C]13 from reaction mixture via semi-preparative HPLC.

FIG. 11B contains images referred to in the examples as Figure S10. Analytical HPLC Spectra for formulated [¹¹C]13.

FIG. 11C contains an image and a table showing prediction of the metabolism sites of 13 with SMART^{Cyp}.

FIG. 12A Distribution of [¹⁸F]mG2N002 in the brain of 10 months old male 3xTg-AD and control mice. The images show an accumulation of [¹⁸F]mG2N002 at the time window of 2-20 min after injection of the radioactivity (120 μCi in 0.05 ml) into the tail vein. The images demonstrate a lower accumulation of [¹⁸F]mG2N002 in AD mouse compared to the control mouse in all investigated brain areas including the striatum, cortex, hippocampus, and thalamus. Axial and sagittal views clearly show the difference between accumulation in different brain areas in AD and control mice.

FIG. 12B Preliminary studies of the binding distribution of [¹⁸F]mG2N002 in female and male 3xTg-AD and control mice. These studies show that accumulation of [¹⁸F]mG2N002 was lower in female AD mice than in female control or male AD mice in all brain areas. Additionally, uptake in male AD mice was lower than in control.

FIG. 13 contains synthetic scheme showing chemical synthesis of compound mG2N003. Reagents and conditions: (a) Pd(dppf)Cl₂, Na₂CO₃, 1,4-dioxane/water, 100° C., 3 h, 52%; (b) 4, methyl isobutyrate, THF, N2, −60° C., 1.5 h, 83%; (c) 6, pyridine, dichloromethane, 25° C., 12 h, 91%; (d) Bu₃Sn, AIBN, toluene, 80° C., 1 h, 31%; (e) LiBH₄, THF, N₂, 60° C., 4 h, 37%; (f) Cs₂CO₃, acetonitrile, 70° C., 12 h, 84%; (g) Zn(CN) 2, Pd(PPh₃)₄, DMF, microwave, 160° C., 30 min, 52%; (h) Na₂CO₃·1.5H₂O₂, acetone, water, rt, 1 h, 76%.

FIG. 14 contains a line plot showing mG2N003 binding potency (see protocol A, Yuan, G. et al. J. Med. Chem. 2022, 65(3), 2593-2609). IC₅₀=578 nM.

### DETAILED DESCRIPTION

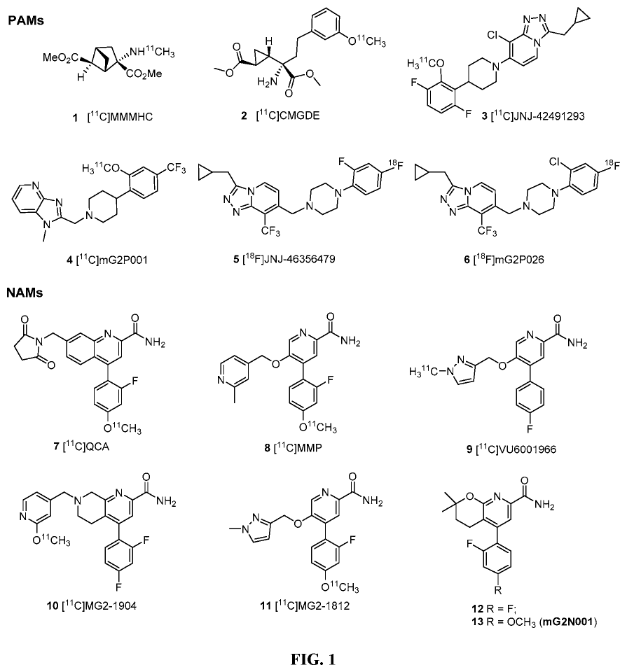

As the most abundant endogenous neurotransmitter in the central nervous system (CNS), glutamate has an important role in regulating several neurological functions in the brain. There are two families of glutamate receptors, namely the ionotropic glutamate receptors (iGluRs) and the metabotropic glutamate receptors (mGluRs). The mGluRs are further divided into three groups based on their sequence homology, pharmacological effects, and distribution. Among them, the group II mGluRs, including mGluR2 and mGluR3, are implicated in the pathologies of several neuropsychiatric disorders, for example, schizophrenia, anxiety, depression, pain, and Alzheimer's disease. Without being bound by any theory, it is believed that mGluR2 and mGluR3 are highly distributed in the forebrain at the presynaptic nerve terminals and activation of these receptors reduces the excessive glutamatergic signaling that is implicated in the pathophysiology of these diseases. Despite the setback of LY2140023, a group II agonist prodrug, in clinical trials for the treatment of schizophrenia, it demonstrated the disease-modifying potential of targeting the mGluR2-focused glutamatergic signaling and emphasized the importance of mGluR2-subtype selectivity for successful drug candidates. As a result, allosteric modulators that bind to the more lipophilic and structurally less conserved seven transmembrane (7-TM) region are developed to afford ligands with more favorable physiochemical properties and enhanced selectivity for mGluR2 binding. Similarly, development of positron emission tomography (PET) radioligands targeting mGluR2 have shifted from the early group II orthosteric ligands, such as the mGluR2/3 antagonists [¹¹C]MMMHC (1) and [¹¹C]CMGDE (2), to the recent allosteric modulators-derived radiotracers, such as the positive allosteric modulators (PAMs) of [¹¹C]JNJ-42491293 (3), [¹¹C]mG2P001 (4), [¹⁸F]JNJ-46356479 (5), and [¹⁸F]mG2P026 (6) (FIG. 1).

As a non-invasive in vivo imaging technique, PET enables the visualization and quantification of mGluR2 under normal and disease conditions as well as the evaluation of target engagement and the dose occupancy studies of drug candidates. Currently, there is no mGluR2 PET tracer for humans. [¹¹C]JNJ-42491293 (3), the only structurally disclosed PET tracer that entered clinical trials, showed unexpected binding in the myocardium and off-target binding in the brain. Besides developing mGluR2 PAM radiotracers for clinical use, it is advantageous to identify negative allosteric modulators (NAMs)-based radiotracers due to their distinct allosteric mode of action and pharmacology. mGluR2 PAMs have both affinity and efficacy cooperativity with glutamate, whereas mGluR2 NAMs show predominantly efficacy cooperativity. [¹¹C]QCA (7, IC₅₀=45 nM) and [¹¹C]MMP (8, IC₅₀=59 nM) (FIG. 1) are disclosed as possible NAM radiotracers. However, these tracers suffered poor brain permeability in rats with a SUVₘₐₓ value of 0.3 and 0.7, respectively. Further studies of these radiotracers in the P-glycoprotein and the breast cancer resistance protein (Pgp-BCRP) knock-out mouse model indicated that they are likely substrates of the efflux pumps on the blood-brain barrier (BBB). QCA (7) was presented in a patent application filed by Merck Research Laboratories in 2013, and the structure-activity relationship was further explored. Compound MMP (8) is an analogous NAM of VU6001966 (9, IC₅₀=78 nM) and has higher brain permeability than QCA (7), prompting its study as a promising PET imaging candidate. However, the ¹¹C-labeled VU6001966 (9) has the same issues as [¹¹C]QCA (7). Since there are no explanations on the structural basis of the poor brain permeability for these NAM tracers, these compounds provide no guidance, in terms of their chemical structures, to the development of improved mGluR2 NAM radiotracers. The NAM tracers [¹¹C]MG-1904 (10, IC₅₀=24 nM) and [¹¹C]MG2-1812 (11, IC₅₀=21 nM) were brain permeable in rats, and they still closely resemble, in terms of structural scaffold, the previously discussed compound VU6001966 (9).

The present disclosure advantageously provides radiotracers on the basis of bicyclic structural scaffold of 3,4-dihydro-2H-pyrano[2,3-b]pyridine. An example of the active compound is 5-(2,4-difluorophenyl)-2,2-dimethyl-3,4-dihydro-2H-pyrano [2,3-b]pyridine-7-carboxamide (12). Compound 12 was reported as a potent mGluR2 NAM (IC₅₀=6.0 nM). As the experimental results show, the compound 12 was successfully radiolabeled to allow for PET imaging. Replacement of the para-fluoride at compound 12 with a phenolic methyl ether advantageously led to active lead compound 5-(2-fluoro-4-methoxyphenyl)-2,2-dimethyl-3,4-dihydro-2H-pyrano[2,3-b]pyridine-7-carboxamide (13) (see FIG. 1), and this retention of activity could not be predicted. The chemical modification introducing methyl ether group allowed the radiolabeling of 13 with [¹¹C]CH₃I via the O-methylation of the corresponding phenol precursor. The synthesis, in vitro characterization and radiolabeling of compounds 12 and 13 as well as the in vivo evaluation of [¹¹C]13 in rats and a non-human primate are described herein. Some embodiments of the related mGluR2 NAM-active compounds with pyrano[2,3-b]pyridine scaffold and the radiotracers prepared on the bases of those compounds (such as the compound of Formula (I) herein) are also disclosed. Some embodiments of pharmaceutical compositions containing these compounds, as well as methods of using these compounds for imaging, diagnosing, and monitoring treatment of mGluR2-mediated diseases are also disclosed.

### Radiotracer Compounds

In some embodiments, the present application provides a compound of Formula (I):

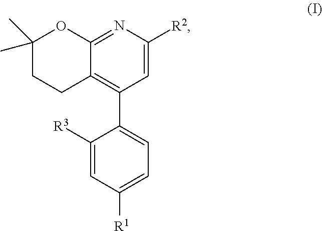

or a pharmaceutically acceptable salt thereof, wherein:
R¹, R², and R³ are each independently selected from halo, CN, C(═O)NH₂, C₁₋₃ alkyl, C₁₋₃ haloalkyl, C₁₋₃ alkoxy, and C₁₋₃ haloalkoxy; and
one of R¹, R², and R³ comprises a radioisotope selected from ¹¹C and ¹⁸F.

In some embodiments, R¹ comprises a radioisotope selected from ¹¹C and ¹⁸F.

In some embodiments, R¹ comprises ¹¹C.

In some embodiments, R¹ comprises ¹⁸F.

In some embodiments, R¹ is selected from ¹⁸F, ¹¹CN, ¹¹C(═O)NH₂, H³₁₁C—, ¹⁸FCH₂CH₂—, ¹¹CH₃O—, ¹⁸FCH₂CH₂O—, ¹⁸FCH₂CH₂CH₂O—, ¹⁸FCD₂O—, and ¹⁸FCH₂O—.

In some embodiments, R¹ is selected from ¹¹CN, ¹¹C(═O)NH₂, H³₁₁C—, and ¹¹CH₃O—.

In some embodiments, R¹ is selected from ¹⁸F, ¹⁸FCH₂CH₂—, ¹⁸FCH₂CH₂O—, ¹⁸FCH₂CH₂CH₂O—, ¹⁸FCD₂O—, and ¹⁸FCH₂O—.

In some embodiments, R¹ is selected from ¹⁸F, ¹¹CN, ¹¹CH₃O—, ¹⁸FCH₂CH₂O—, ¹⁸FCH₂CH₂CH₂O—, ¹⁸FCD₂O—, and ¹⁸FCH₂O—.

In some embodiments, R¹ is selected from ¹⁸F and ¹¹CH₃O—. In some embodiments, R¹ is ¹⁸F. In some embodiments, R¹ is ¹¹CH₃O—.

In some embodiments, the compound of Formula (I) has formula:

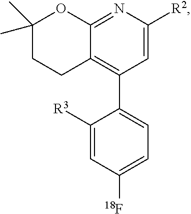

or a pharmaceutically acceptable salt thereof.

In some embodiments, the compound of Formula (I) has formula:

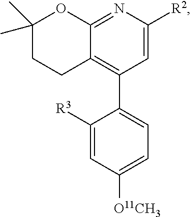

or a pharmaceutically acceptable salt thereof.

In some embodiments, R² is selected from halo, CN, C(═O)NH₂, C₁₋₃ alkyl, and C₁₋₃ haloalkyl.

In some embodiments, R² is selected from halo, CN, and C(═O)NH₂.

In some embodiments, R² is selected from CN and C(═O)NH₂.

In some embodiments, R² is CN.

In some embodiments, R² is C(═O)NH₂.

In some embodiments, the compound of Formula (I) has formula:

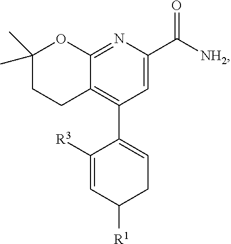

or a pharmaceutically acceptable salt thereof.

In some embodiments, R³ is selected from halo, CN, C(═O)NH₂, C₁₋₃ alkyl, C₁₋₃ haloalkyl, C₁₋₃ alkoxy, and C₁₋₃ haloalkoxy.

In some embodiments, R³ is selected from halo, CN, C₁₋₃ alkoxy, and C₁₋₃ haloalkoxy.

In some embodiments, R³ is selected from halo, C₁₋₃ alkoxy, and C₁₋₃ haloalkoxy. In some embodiments, R³ is selected from C₁₋₃ alkoxy, and C₁₋₃ haloalkoxy.

In some embodiments, R³ is selected from halo and C₁₋₃ alkoxy. In some embodiments, R³ is halo. In some embodiments, R³ is C₁₋₃ alkoxy.

In some embodiments, the compound of Formula (I) has formula:

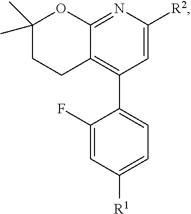

or a pharmaceutically acceptable salt thereof.

In some embodiments:
R² is selected from halo, CN, and C(═O)NH₂; and
R³ is selected from halo, C₁₋₃ alkoxy, and C₁₋₃ haloalkoxy.

In some embodiments:
R² is selected from CN and C(═O)NH₂; and
R³ is selected from halo and C₁₋₃ alkoxy.

In some embodiments, R³ is halo (e.g., F, Br, I, or Cl).

In some embodiments, the compound of Formula (I) has formula:

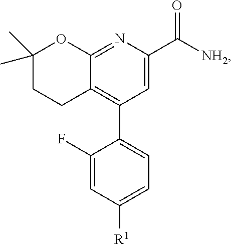

or a pharmaceutically acceptable salt thereof.

In some embodiments, the compound of Formula (I) is selected from any one of the following compounds:

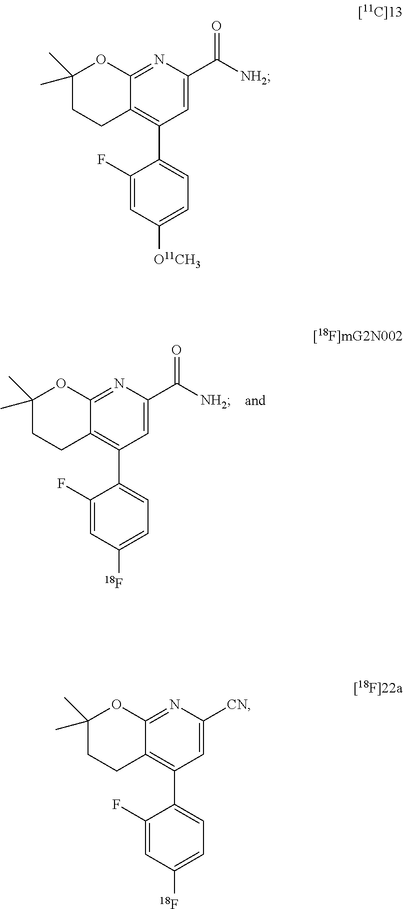

or a pharmaceutically acceptable salt thereof.

In some embodiments, the compound of Formula (I) has formula:

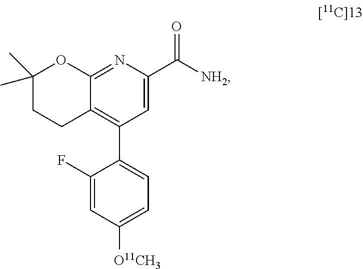

or a pharmaceutically acceptable salt thereof.

In some embodiments, the compound of Formula (I) has formula:

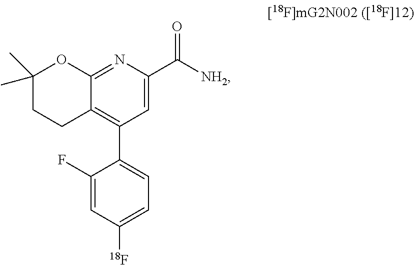 [SMILES: CC1(C)CCc2c(-c3ccc([18F])cc3F)cc(C(N)=O)nc2O1]

or a pharmaceutically acceptable salt thereof.

In some embodiments, the compound of Formula (I) is selected from any one of the following compounds:

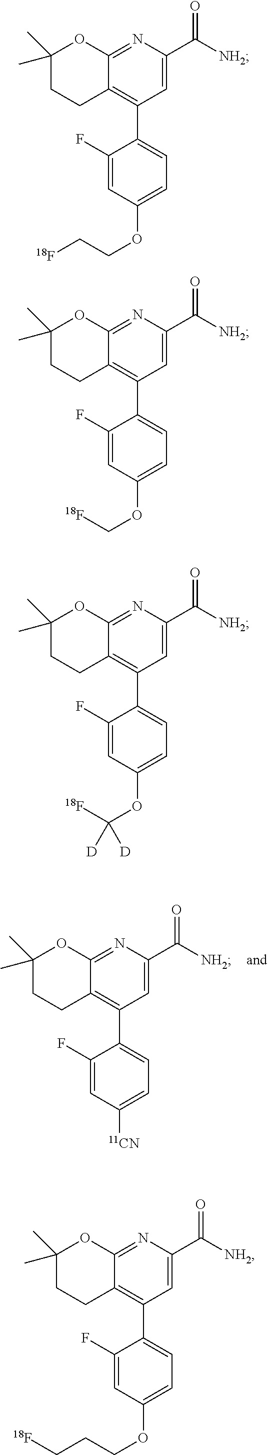

or a pharmaceutically acceptable salt thereof.

In some embodiments, R² comprises a radioisotope selected from ¹¹C and ¹⁸F.

In some embodiments, R² comprises ¹¹C.

In some embodiments, R² comprises ¹⁸F.

In some embodiments, R² is selected from ¹⁸F, ¹¹CN, ¹¹C(═O)NH₂, H³₁₁C—, ¹⁸FCH₂CH₂—, ¹¹CH₃O—, ¹⁸FCH₂CH₂O—, ¹⁸FCH₂CH₂CH₂O—, ¹⁸FCD₂O—, and ¹⁸FCH₂O—.

In some embodiments, R² is selected from ¹¹CN, ¹¹C(═O)NH₂, H³₁₁C—, and ¹¹CH₃O—. In some embodiments, R² is selected from ¹⁸F, ¹⁸FCH₂CH₂—, ¹⁸FCH₂CH₂O—, ¹⁸FCH₂CH₂CH₂O—, ¹⁸FCD₂O—, and ¹⁸FCH₂O—.

In some embodiments, R² is selected from ¹¹CN and ¹¹C(═O)NH₂. In some embodiments, R² is ¹¹CN. In some embodiments, R² is ¹¹C(═O)NH₂.

In some embodiments, the compound of Formula (I) has formula:

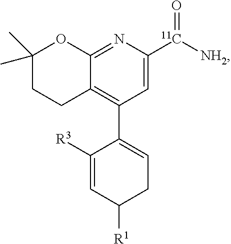

or a pharmaceutically acceptable salt thereof.

In some embodiments, R² and R³ are each independently selected from halo, C₁₋₃ alkoxy, and C₁₋₃ haloalkoxy.

In some embodiments, R² and R³ are each independently selected from halo and C₁₋₃ alkoxy. In some embodiments, R² is selected from halo and C₁₋₃ alkoxy. In some embodiments, R³ is selected from halo and C₁₋₃ alkoxy.

In some embodiments, R² and R³ are each independently halo. In some embodiments, R² is halo (e.g., F, Cl, I, or Br). In some embodiments, R³ is halo (e.g., F, Cl, I, or Br). In some embodiments, R² is C₁₋₃ alkoxy. In some embodiments, R³ is C₁₋₃ alkoxy.

In some embodiments, the compound of Formula (I) has formula:

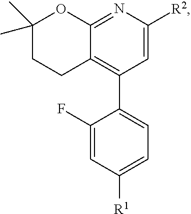

or a pharmaceutically acceptable salt thereof.

In some embodiments, the compound of Formula (I) has formula:

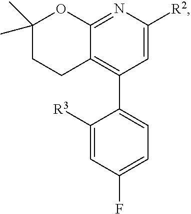

or a pharmaceutically acceptable salt thereof.

In some embodiments, the compound of Formula (I) has formula:

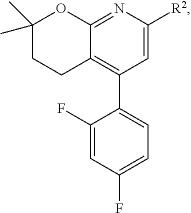

or a pharmaceutically acceptable salt thereof.

In some embodiments, the compound of Formula (I) is selected from any one of the following compounds:

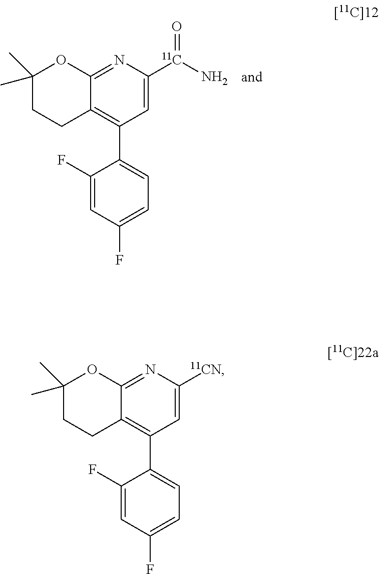

or a pharmaceutically acceptable salt thereof.

In some embodiments, the compound of Formula (I) has formula:

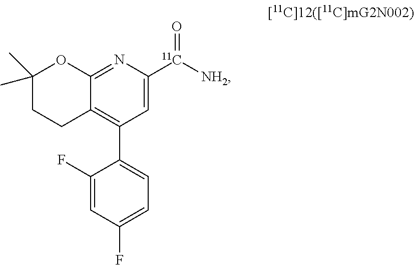 [SMILES: CC1(C)CCc2c(-c3ccc(F)cc3F)cc([11C](N)=O)nc2O1]

or a pharmaceutically acceptable salt thereof.

In some embodiments, R³ comprises a radioisotope selected from ¹¹C and ¹⁸F.

In some embodiments, R³ comprises ¹¹C.

In some embodiments, R³ comprises ¹⁸F.

In some embodiments, R³ is selected from ¹⁸F, ¹¹CN, ¹¹C(═O)NH₂, H³₁₁C—, ¹⁸FCH₂CH₂—, ¹¹CH₃O—, ¹⁸FCH₂CH₂O—, ¹⁸FCH₂CH₂CH₂O—, ¹⁸FCD₂O—, and ¹⁸FCH₂O—.

In some embodiments, R³ is selected from ¹⁸F, ¹⁸FCH₂CH₂—, ¹⁸FCH₂CH₂O—, ¹⁸FCH₂CH₂CH₂O—, ¹⁸FCD₂O—, and ¹⁸FCH₂O—. In some embodiments, R³ is selected from ¹¹CN, ¹¹C(═O)NH₂, H³₁₁C—, and ¹¹CH₃O—.

In some embodiments, R³ is selected from ¹⁸F, ¹¹CH₃O—, ¹⁸FCH₂CH₂O—, ¹⁸FCH₂CH₂CH₂O—, ¹⁸FCD₂O—, and ¹⁸FCH₂O—. In some embodiments, R³ is ¹⁸F. In some embodiments, R³ is ¹¹CH₃O—. In some embodiments, R³ is ¹⁸FCH₂CH₂O—. In some embodiments, R³ is ¹⁸FCH₂O—. In some embodiments, R³ is ¹⁸FCH₂CH₂CH₂O—.

In some embodiments, R³ is ¹⁸FCD₂O—.

In some embodiments, the compound of Formula (I) has formula:

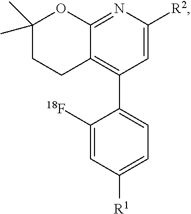

or a pharmaceutically acceptable salt thereof.

In some embodiments, the compound of Formula (I) has formula:

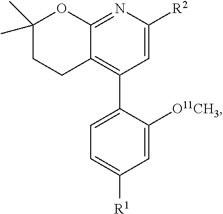

or a pharmaceutically acceptable salt thereof.

In some embodiments, R¹ is selected from halo, C₁₋₃ alkoxy, and C₁₋₃ haloalkoxy. In some embodiments, R¹ is halo. In some embodiments, R¹ is C₁₋₃ alkoxy. In some embodiments, R¹ is C₁₋₃ haloalkoxy. In some embodiments, R¹ is selected from halo and C₁₋₃ alkoxy.

In some embodiments, R² is selected from halo, CN, and C(═O)NH₂. In some embodiments, R² is selected from CN and C(═O)NH₂. In some embodiments, R² is CN. In some embodiments, R² is C(═O)NH₂.

In some embodiments:
R¹ is selected from halo, C₁₋₃ alkoxy, and C₁₋₃ haloalkoxy; and
R² is selected from halo, CN, and C(═O)NH₂.

In some embodiments:
R¹ is selected from halo and C₁₋₃ alkoxy; and
R² is selected from CN and C(═O)NH₂.

In some embodiments, R¹ is halo.

In some embodiments, the compound of Formula (I) is selected from any one of the following compounds:

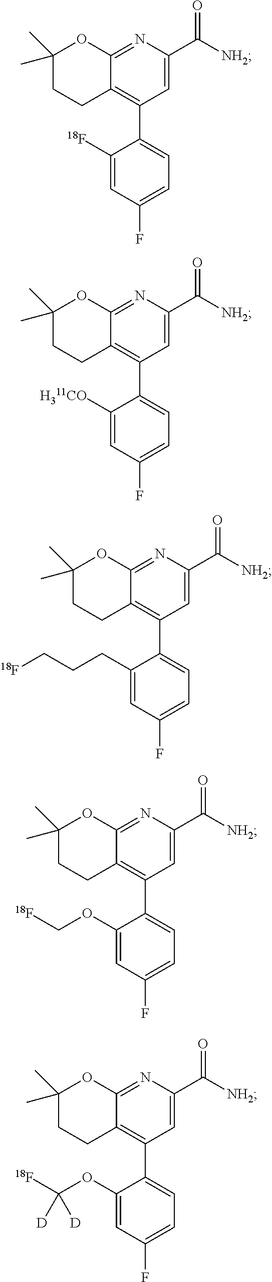

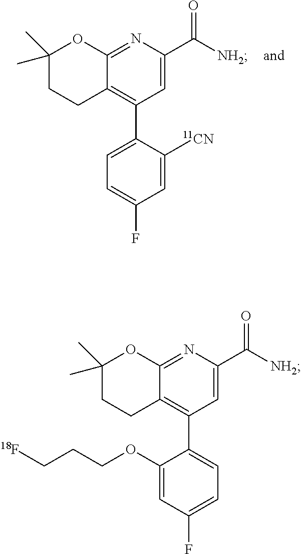

or a pharmaceutically acceptable salt thereof.

In some embodiments, the compound of Formula (I) is selected from any one of the following compounds:

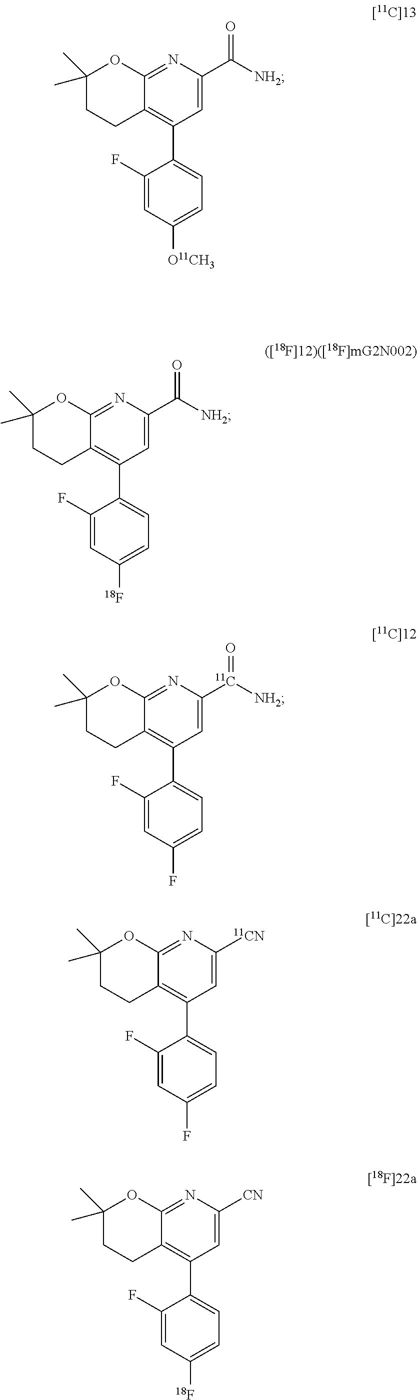

or a pharmaceutically acceptable salt thereof.

In some embodiments, the compound of Formula (I) is selected from any one of the following compounds:

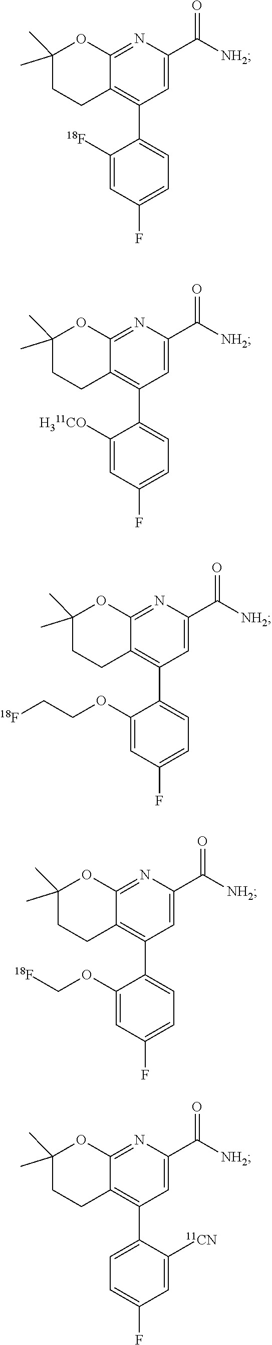

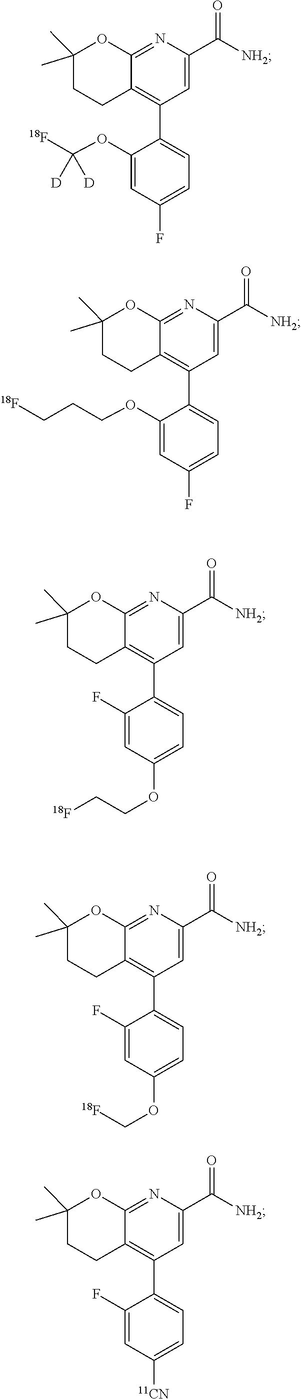

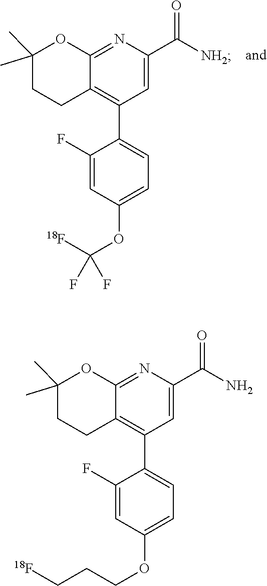 [SMILES: CC1(C)CCc2c(-c3ccc(OC(F)(F)[18F])cc3F)cc(C(N)=O)nc2O1 ; CC1(C)CCc2c(-c3ccc(OCCC[18F])cc3F)cc(C(N)=O)nc2O1]

or a pharmaceutically acceptable salt thereof.

### Pharmaceutically Acceptable Salts

In some embodiments, a salt of any one of the compounds of the present disclosure is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxy benzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

### Methods of Use

In one general aspect, the present application relates to compounds of formula (I) useful in imaging techniques, diagnosing and monitoring treatment of various diseases and conditions described herein. Such compounds are labeled in so far as each compound includes at least one ¹⁸F radioisotope or at least one ¹¹C isotope.

PET has become an important clinical diagnostic and research modality, and also a valuable technology in drug discovery and development. PET offers picomolar sensitivity and is a fully translational technique that requires specific probes radiolabeled with a usually short-lived positron-emitting radionuclide. Carbon-11 (radioactive half-life (t_{1/2})=20.4 min) and fluorine-18 (t_{1/2}=109.7 min) are the most commonly used radionuclides in PET imaging. PET has provided the capability of measuring biological processes at the molecular and metabolic levels in vivo by the detection of the photons formed as a result of the annihilation of the emitted positrons.

As a noninvasive medical and molecular imaging technique and a powerful tool in neurological research, PET offers the possibility of visualizing and analyzing the target receptor expression under physiological and pathophysiological conditions. PET has often been used to detect disease-related biochemical changes before the disease-associated anatomical changes can be found using standard medical imaging modalities.

Moreover, PET tracers serve as invaluable biomarkers during the clinical development of potential therapeutics, in which the receptor occupancy of potential drug candidates in the brain is measured. In vivo receptor occupancy can help to answer many vital questions in the drug discovery and development process, such as whether potential drugs reach their molecular targets, the relationship between therapeutic dose and receptor occupancy, the correlation between receptor occupancy and plasma drug levels, and the duration of time the drug remains at its target.

Despite the great wealth of information that such probes can provide, the potential of PET strongly depends on the availability of suitable PET radiotracers. However, existing tracer discussed earlier suffer from serious drawbacks, including off-target binding, low BBB-penetration, and undesirable interaction with brain efflux pumps. The compounds within the present claims (containing ¹⁸F or ¹¹C atom) cross the BBB quickly and are mainly accumulated, e.g., in striatum, thalamus, hypothalamus, hippocampus, cerebellum, cortex, and/or putamen, which were reported as the mGluR2-rich regions of the brain, do not engage in off-target binding, and do not interact with brain efflux pumps. As such, the mGluR2-modulating compounds within the present claims are excellent PET-detectable tracers for imaging 20 mGluR2 in the brain. The compounds provide the capability of measuring biological processes involving mGluR2 in the brain at a molecular level in vivo. The compounds are therefore useful as a clinical diagnostic and research modality (e.g., in drug discovery and development) to advance treatment and/or prevention of neurological and psychiatric disorders.

L-Glutamate is the most abundant excitatory neurotransmitter in the central nervous system (CNS) of vertebrates and probably mediates more than 50% of all synapses. Two major classes of receptors, ionotropic glutamate receptors (iGluRs) and metabotropic glutamate receptors (mGluRs), as well as transporters are involved in glutamate signaling. iGluRs, including the N-methyl-d-aspartate (NMDA), a-amino-3-hydroxy-5-methyl-4-isoxazole-propionate (AMPA), and kainite receptors are ligand-gated ion channels that mediate fast synaptic transmission. The mGluRs modulate the presynaptic glutamate release and/or postsynaptic effects of glutamate. mGluRs belong to class C of the G-protein-coupled receptor (GPCR) super family, which can be further divided into three subgroups including eight known receptor sub-types (group I: mGluR1 and mGluR5, group II: mGluR2 and mGluR3, and group III: mGluR4, mGluR6, mGluR7, and mGluR8) based on their structural similarity, ligand specificity, and preferred coupling mechanism. mGluRs are involved in glutamate signaling in almost every excitatory synapse in CNS, and they have distinctive biodistribution in CNS depending on subtypes and subgroups.

mGluR2 is a group II metabotropic glutamate receptor that is widely expressed in the forebrain and localized presynaptically, where it negatively modulates glutamate and GABA release. Initial research and drug discovery efforts had focused on pharmacological ligands for mGluR2, which had largely been competitive orthosteric ligands including agonists and antagonists. These competitive orthosteric ligands possess extremely high potencies or binding affinities for the group, but poor selectivity within the group (e.g., between the mGluR2 and mGluR3 receptors). A number of extremely potent mGluR2 selective agonists have been published in literature and developed for the treatment of anxiety and schizophrenia in preclinical and clinical studies. While normalization of glutamate levels through the use of mGluR2 agonists had shown comparable efficacy to conventional antipsychotic drugs for the treatment of schizophrenia, the preclinical studies revealed that the antipsychotic effect of mGluR2 agonists was absent in mGluR2 knockout mice but not mGluR3 knockout mice, suggesting the antipsychotic effects might be mediated via the mGluR2 but not mGluR3 receptor and even the effect of mGluR2 and mGluR3 might be different/opposite.

Because of the high degree of homology at the orthosteric sites of group II mGluRs, selective mGluR2 agonists have been difficult to design. Recently developed allosteric modulators have changed glutamate related drug development. Allosteric modulators are small molecules capable of enhancing agonist or antagonist mediated receptor activity while possessing no or less intrinsic agonist or antagonist activity. Relative to classical mGluR agonists and antagonists, the PAMs and NAMs offer improved selectivity versus other mGluRs and chemical tractability, and may reduce receptor desensitization specificity.

It is important to develop allosteric mGluR2-selective ligands for the diagnosis and treatment of neurological and psychiatric disorders. In recent years, several pharmaceutical companies and research groups have focused on developing PAMs and NAMs of mGluR2 as therapeutic drugs for different neurological conditions. Targeting mGluR2 with allosteric modulators has advantages over orthosteric ligands such as improved selectivity and better tolerability, which may offer enhanced therapeutic effects as well as improved side-effect profiles. It has been shown that enhancement or inhibition of mGluR functions has different biological response, which can be related to different diseases (such as those described herein). For example, it has been suggested that PAMs of mGluR2 can be related to therapeutic approaches of pain, schizophrenia, and drug abuse; while NAMs can be related to the therapeutics of cognitive disorders like AD. In some embodiments, either PAM or NAM of mGluR2 can be used to treat any one of the disorders described herein.

In some embodiments, the present disclosure provides a method of modulating (e.g., positively allosterically modulating or negatively allosterically modulating) mGluR2 in a cell, the method comprising contacting the cell with an effective amount of a compound of the present disclosure (e.g., Formula (I)), or a pharmaceutically acceptable salt thereof, or a pharmaceutical composition comprising same. In some embodiments, the modulating comprises binding, inhibiting, or activating, or any combination of the foregoing. In some embodiments, the contacting occurs in vitro, in vivo, or ex vivo. In some embodiments, the cell is a brain cell (e.g., a neuron or a glial cell). In some embodiments, the modulation is selective with respect to mGluR2, as opposed to other mGluR receptors (e.g., the modulation is 10×, 20×, 50×, 100×, or 1000× more selective with respect to mGluR2). In some embodiments, the present disclosure provides a method of modulating (e.g., as described above) mGluR2 in a subject, the method comprising administering to the subject an effective amount of a compound of the present disclosure (e.g., Formula (I)), or a pharmaceutically acceptable salt thereof, or a pharmaceutical composition comprising same.

In some embodiments, the present disclosure provides a method of identifying and quantifying mGluR2 density in the brain of a subject. This may be attained, for example, by imaging the brain. A method of imaging the brain comprises (i) administering to the subject an effective amount of a compound of Formula (I), or a pharmaceutically acceptable salt thereof, or a pharmaceutical composition comprising same; (ii) waiting a time sufficient to allow the compound to accumulate in the brain to be imaged (e.g., 1 min, 5 min, 10 min, 10 min, 30 min, or up to 60 min), and (iii) imaging the brain with an imaging technique. Without being bound by any theory, since ¹⁸F or ¹¹C within the compound of Formula (I) is a positron emitting radioisotope, the suitable imaging techniques include positron emission tomography (PET) and its modifications including high-energy gamma imaging devices. As such, the imaging technique may be selected from positron emission tomography (PET) imaging, positron emission tomography with computer tomography (PET/CT) imaging, and positron emission tomography with magnetic resonance (PET/MRI) imaging, as well as other suitable methods.

In some embodiments, the present disclosure provides a method of diagnosing (or early detection) a psychiatric or a neurological disorder (e.g., psychiatric or neurological disorder in the pathology of which mGluR2 is implicated, as described herein) in a subject, the method comprising (i) administering to the subject an effective amount of a compound of Formula (I), or a pharmaceutically acceptable salt thereof, or a pharmaceutical composition comprising same; (ii) waiting a time sufficient to allow the compound to accumulate in the brain to be imaged (e.g., 1 min, 5 min, 10 min, 15 min, or 30 min), and (iii) imaging the brain with an imaging technique. The method may also comprise comparing images obtained from subjects exhibiting the symptoms of the disease or condition with the images obtained from healthy subjects. For the treating physician or a diagnostician, this comparison may reveal important information aiding in the diagnosis. In one example, loss or overabundance of mGluR2 receptors in the brain of the subject may be indicative of a neurodegenerative disease (e.g., Alzheimer's disease or Parkinson's disease) or a psychiatric disease (e.g., schizophrenia or depression), or a related condition as described herein.

In some embodiments, the mGluR2-selective PET radiotracers of Formula (I) within the present claims are useful to study the role of mGluR2 in health and disease conditions. In some embodiments, the present disclosure provides a method of supporting the clinical development of potential therapeutics, in which the receptor occupancy of potential drug candidates such as mGluR2 allosteric modulators in the brain is measured. In vivo receptor occupancy can help to answer many vital questions in the drug discovery and development process such as whether potential drugs reach their molecular targets, the relationship between therapeutic dose and receptor occupancy, the correlation between receptor occupancy and plasma drug levels, and the duration of time the drug remains at its target. In some embodiments, the present disclosure provides a method (e.g., an assay) of identifying a compounds that modulates mGluR2 in a brain of a subject, the method comprising (i) administering a compound of Formula (I), or a pharmaceutically acceptable salt thereof, in combination with a test compound, or a pharmaceutically acceptable salt thereof, to a subject; (ii) waiting a time sufficient to allow the compound to accumulate in the brain to be imaged; (iii) imaging the brain with an imaging technique; and (iv) determining whether the image obtain in step iii) is different from an image of the brain obtain after administering the compound of Formula (I), or a pharmaceutically acceptable salt thereof, alone; wherein the difference observed in step iv) is indicative of competitive binding to mGluR2 in the brain between the test compound and the compound of Formula (I). In some embodiments, the method is a high-throughput screening assay.

In yet other embodiments, the present disclosure provides a method of monitoring treatment of a psychiatric or a neurological disorder (e.g., a psychiatric or a neurological disorder in which mGluR2 is implicated) in a subject, the method comprising (i) administering to the subject an effective amount of a compound of Formula (I), or a pharmaceutically acceptable salt thereof, or a pharmaceutical composition comprising same, (ii) waiting a time sufficient to allow the compound of Formula (I) administered in step (i) to accumulate in a brain of the subject (e.g., 5 min, 15 min, or 30 min); (iii) imaging the brain of the subject with an imaging technique; (iv) administering to the subject a therapeutic agent in an effective amount to treat the psychiatric or the neurological disorder. In one example, Aducanumab, Donepezil, Rivastigmine, Galantamine, Memantine, Suvorexant, or an experimental drug substance for treating AD may be administered to a subject undergoing treatment of AD. In another example, levodopa (L-dopa), carbidopa, safinamide, dopamine agonists (e.g., ropinirole, pramipexole, rotigotine), amantadine, trihexyphenidyl, benztropine, selegiline, rasagiline, tolcapone, entacapone, or an experimental drug substance for treating PD may be administered to a subject undergoing treatment of PD. In yet another example, sertraline, fluoxetine, citalopram, paroxetine, bupropion, or an experimental drug substance for treating depression can be administered to the subject undergoing treatment of depression. In yet another example, aripiprazole, asenapine, cariprazine, clozapine, lloperidone, lumateperonee, or experimental drug substance for treating schizophrenia may be administered to the subject undergoing treatment of schizophrenia.

In some embodiments, the method further includes step (v) after (iv), comprising administering to the subject an effective amount of a compound of Formula (I), or a pharmaceutically acceptable salt thereof; (vi) waiting a time sufficient to allow the compound of Formula (I) administered in step (v) to accumulate in the brain of the subject (e.g., 5 min, 15 min, or 30 min); (vii) imaging the brain of the subject with an imaging technique; and (viii) comparing the image of step (iii) and the image of step (vii). In one example, attaining abundance or overabundance of mGluR2 receptors in the brain of the subject, as determined by comparing the images, is indicative of successful treatment of the psychiatric or the neurological disorder. Suitable examples of diseases the treatment of which can be monitored according to the methods of the present disclosure include any of the diseases described herein. One particular example is schizophrenia. Other suitable examples include AD, PD, pain, psychosis, epilepsy, anxiety, depression, drug abuse, smoking cessation, dyskinesia, Lewy body disease, Prion disease, motor neuron disease (MND), and Huntington's disease. In some embodiments, the neurological disorder associated with mGluR2 is selected from Alzheimer's disease, Parkinson's disease, dyskinesia, Lewy body disease, Prion disease, motor neuron disease (MND), and Huntington's disease. In some embodiments, the psychiatric disorder associated with mGluR2 is selected from schizophrenia, psychosis, anxiety, bipolar disorder, depression, drug abuse, addiction, pain, smoking cessation, and epilepsy.

### Compositions, Formulations, and Mutes of Administration

The present application also provides pharmaceutical compositions comprising an effective amount of a compound of the present disclosure (e.g., Formula (I)) disclosed herein, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any 25 combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, a compound of the present disclosure (e.g., a compound of Formula (I)) is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound (e.g., Formula (I)) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of Formula (I) is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, “halo” refers to F, Cl, Br, or I. In some embodiments, a halo is F, Cl, or Br.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified. Any atom identified in the compounds herein that is not specifically designated as radioisotope is present at is natural isotopic abundance.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the mGluR2 with a compound of the invention includes the administration of a compound of the present invention to an individual or patient, such as a human, having mGluR2, as well as, for example, introducing a compound of the invention into a sample containing a cellular or purified preparation containing the mGluR2.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

As used herein, the term “radioisotope” refers to an atom having an atomic mass or mass number different from the atomic mass or mass number typically found in nature (i.e., naturally occurring).

As used herein, the term “isotopic enrichment factor” refers to the ratio between the isotopic abundance and the natural abundance of a specified isotope.

“D” and “d” both refer to deuterium. A compound of the present disclosure has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

“¹⁸F” refers to the radioisotope of fluorine having 9 protons and 9 neutrons. “F” refers to the stable isotope of fluorine having 9 protons and 10 neutrons (i.e., the “¹⁹F isotope”). A compound of the present disclosure has an isotopic enrichment factor for each designated ¹⁸F atom of at least 3500 (52.5% ¹⁸F incorporation at each designated ¹⁸F atom), at least 4000 (60% ¹⁸F incorporation), at least 4500 (67.5% ¹⁸F incorporation), at least 5000 (75% ¹⁸F), at least 5500 (82.5% ¹⁸F incorporation), at least 6000 (90% ¹⁸F incorporation), at least 6333.3 (95% ¹⁸F incorporation), at least 6466.7 (97% ¹⁸F incorporation), at least 6600 (99% ¹⁸F incorporation), or at least 6633.3 (99.5% ¹⁸F incorporation).

“¹¹C” refers to the radioisotope of carbon having 6 protons and 5 neutrons. “C” refers to the stable isotope of carbon having 6 protons and 6 neutrons (i.e., the “¹²C isotope”). A compound of the present disclosure has an isotopic enrichment factor for each designated ¹¹C atom of at least 3500 (52.5% ¹¹C incorporation at each designated ¹¹C atom), at least 4000 (60% ¹¹C incorporation), at least 4500 (67.5% ¹¹C incorporation), at least 5000 (75% ¹¹C), at least 5500 (82.5% ¹¹C incorporation), at least 6000 (90% ¹¹C incorporation), at least 6333.3 (95% ¹¹C incorporation), at least 6466.7 (97% ¹¹C incorporation), at least 6600 (99% ¹¹C incorporation), or at least 6633.3 (99.5% ¹¹C incorporation).

### EXAMPLES

The following examples are illustrative and not limiting.

Example 1—Synthesis and characterization of 5-(2-fluoro-4-[¹¹C]methoxyphenyl)-2,2-dimethyl-3,4-dihydro-2H-pyrano[2,3-b]pyridine-7-carboxamide as a PET imaging ligand for metabotropic glutamate receptor 2

### Results and Discussion

Chemistry. Syntheses of compounds 12, 13 and the phenolic precursor 24 are shown in Scheme 1. The syntheses started from the Wittig reaction between aldehyde 14 and phosphorous ylide 15 to give compound 16. Hydrogenation of compound 16 under 40 psi of hydrogen at room temperature led to compound 17, which was used in the subsequent Suzuki coupling reaction with the boronic acid species 18a-18c to furnish compounds 19a-19c. The ester groups in compounds 19a-19c were converted to tertiary alcohol moieties in compounds 20a-20c at 0° C. in the presence of a Grignard reagent. After cyclization of the tertiary alcohols under basic conditions, aryl chlorides 21a-21c were obtained, which were cyanated with Zn(CN)₂ in a microwave reactor to give aryl nitriles 22a-22c. Finally, hydrolysis of 22a and 22b led to compounds 12 and 13, whereas compound 22c was deprotected before hydration to afford the radiolabeling precursor 24.

During the syntheses of these compounds, several modifications were made to the previous methods. First, the more reactive 4-iodo-2,6-dichloronicotinaldehyde (14) instead of 4-bromo-2,6-dichloronicotinaldehyde was used as starting material.

Second, compound 16 was hydrogenated to 17 prior to the Suzuki coupling with 18a-18c. bird, the carboxamide group in compounds 12, 13 and 24 was introduced by a microwave-assisted cyanation with Zn(CN)₂ at 160° C. for 30 min followed by hydration with Na₂CO₃·1.5H₂O₂. Previously, this function group was installed via the palladium-catalyzed esterification of aryl chlorides 21a-21c under 50 psi of carbon monoxide at 80° C. for 30 h and subsequent amidation with ammonia. The new synthetic methods in scheme 1 were robust and gave compounds 12, 13 and 24 with overall yields of 2.7%, 7.1%, and 1.2%, respectively, whereas the yield of compound 12 was not disclosed previously.

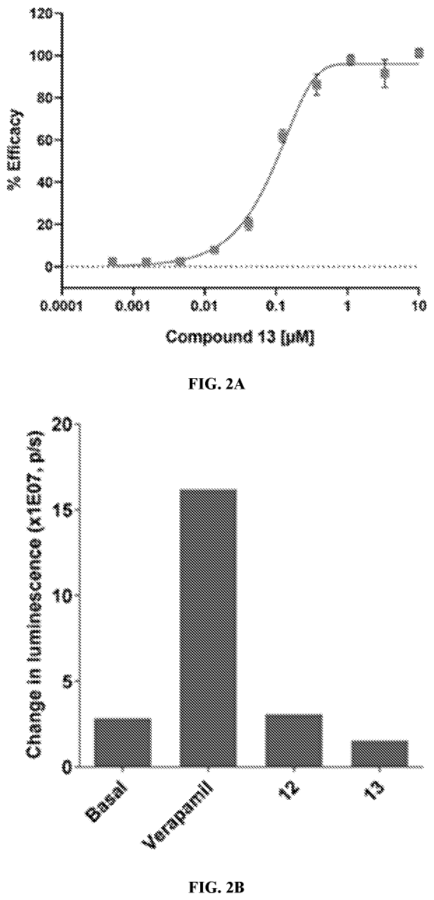

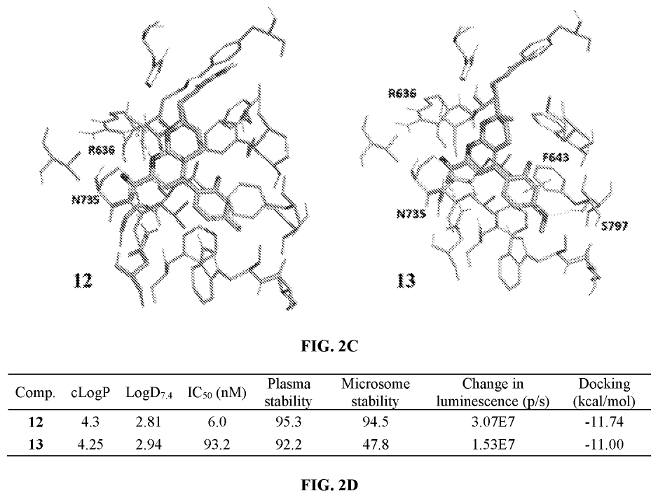

Pharmacology and Physiochemical Properties. As previously disclosed, compound 12 had a potent mGluR2 negative allosteric modulatory activity (IC₅₀=6 nM). The IC₅₀ value was determined by measuring the inhibition of glutamate-induced calcium mobilization in Chinese Hamster Ovary (CHO) cells expressing recombinant human mGluR2. Herein, the modulatory activity of compound 13 was tested by monitoring the cAMP modulation using the DiscoverX HitHunter cAMP XS+ assay. The CHO cells expressing recombinant human mGluR2 were used. Compound 13 was determined as a potent mGluR2 NAM with an IC₅₀ value of 93.2 nM (FIG. 2A).

Besides the mGluR2 binding, the physicochemical properties of compounds 12 and 13 were also characterized using the previously described assays. The assays assessed their lipophilicity, plasma stability, liver microsome stability, and their effect on recombinant human P-glycoprotein (Pgp). The lipophilicity of 12 and 13 was initially predicted in ChemDraw 16.0 with a c Log P value of 4.3 and 4.25, respectively (Table 1). This property was further tested using the “shake flask method” to give a Log D_{7.4} value of 2.81 and 2.94 for compounds 12 and 13, respectively, which are in the preferred range of 1.0-3.5 for brain permeable compounds (Table 1). Compound 12 showed excellent stabilities in rat plasma and rat liver microsome assays (>92%), whereas compound 13 had excellent rat plasma stability (94.5%) but moderate rat liver microsome stability (47.8%, Table 1). In addition, compounds 12 and 13 were evaluated by the Pgp-Glo™ assay. The assay detects the effects of a tested compound toward recombinant human Pgp protein in a cell membrane fraction. If the compound is a transport substrate of Pgp, it stimulates the Pgp ATPase reaction, resulting in ATP consumption and subsequent decrease of the luciferase-generated luminescent signal. The basal Pgp ATPase activity was measured by the change in luminescence between sodium orthovanadate (Na₃VO₄)-treated controls and untreated samples. Verapamil, a known transport substrate of Pgp, was used as a positive control. As shown in FIG. 2B and Table 1, the change in luminescence for compounds 12 and 13 was similar to that of the basal condition, suggesting neither compound 12 nor compound 13 had any effect with this protein. Therefore, compounds 12 and 13 were used as starting materials for making PET imaging ligands.

To probe the ligand-protein binding of compounds 12 and 13, mGluR2 homology model for NAMs was prepared via YASARA and the molecular docking was performed. As shown in FIGS. 2C, compounds 12 and 13 adopted similar binding poses in the allosteric binding pocket. For both compounds, the oxygen atom in the carboxamide sidechain forms a hydrogen bond with Asn735 and the nitrogen atom in the carboxamide side chain forms a hydrogen bond with R636. Moreover, compound 13 forms an extra hydrogen bond with its methyl ether oxygen atom to Ser797 and an additional n-n stacking with its phenyl ring toward Phe643. The docking score of compounds 12 and 13 were −11.74 kcal/mol and −11.00 kcal/mol, respectively, indicating their potential nanomolar binding affinity for mGluR2.

Radiochemistry. Although compound 12 had better pharmacological and physicochemical properties than compound 13, radiolabeling of this compound required a different synthetic strategy.

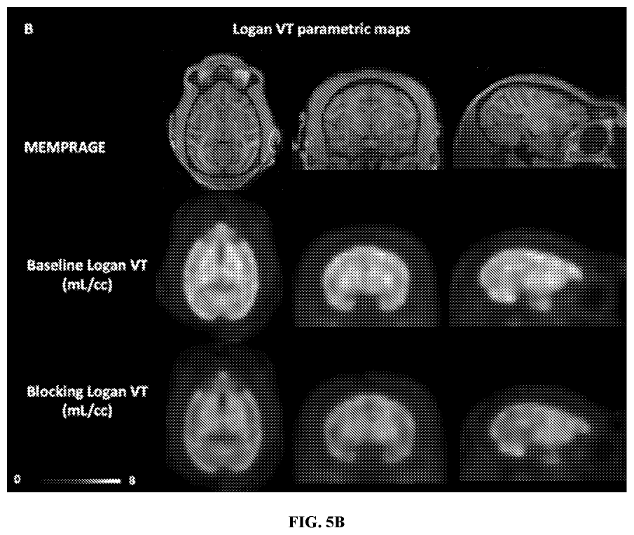

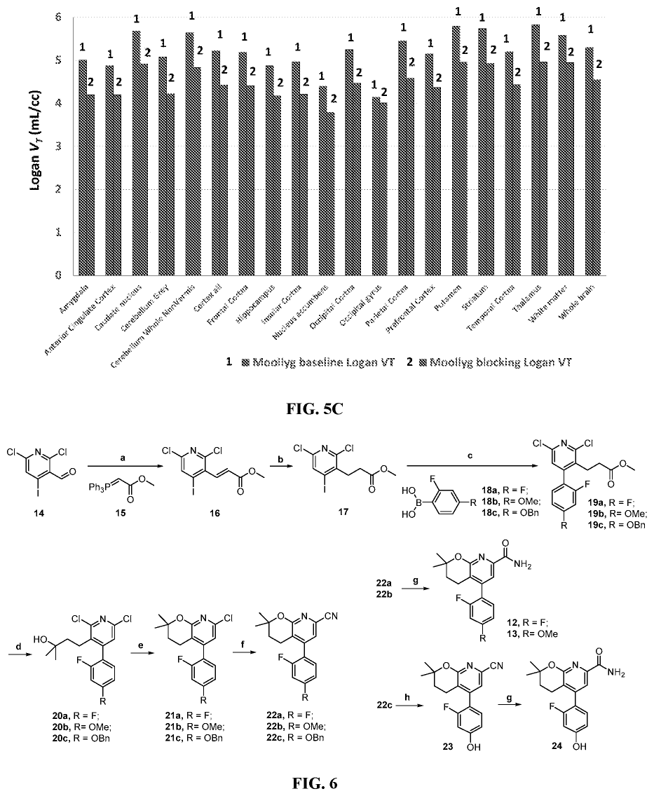

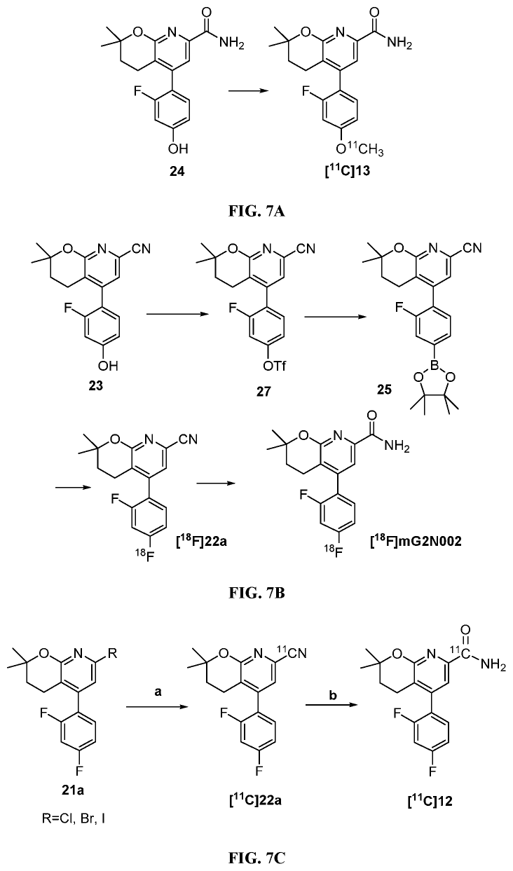

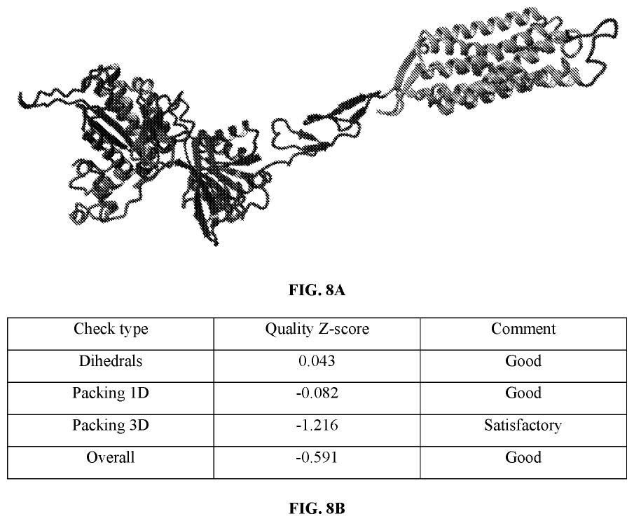

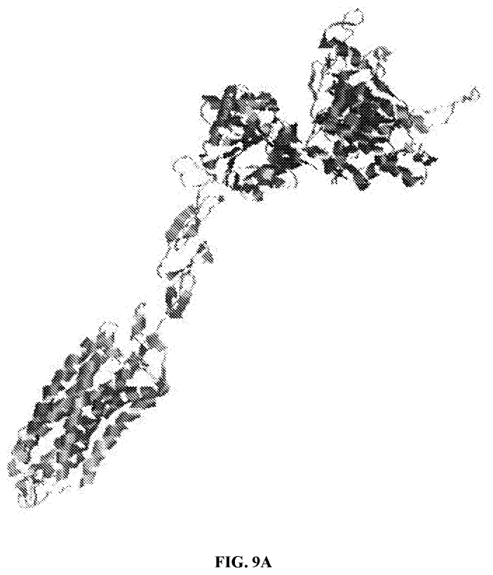

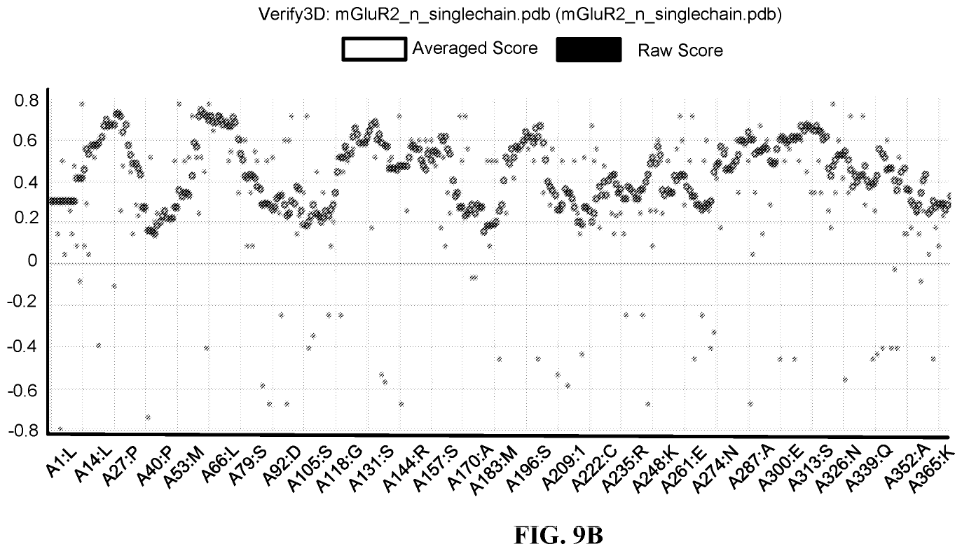

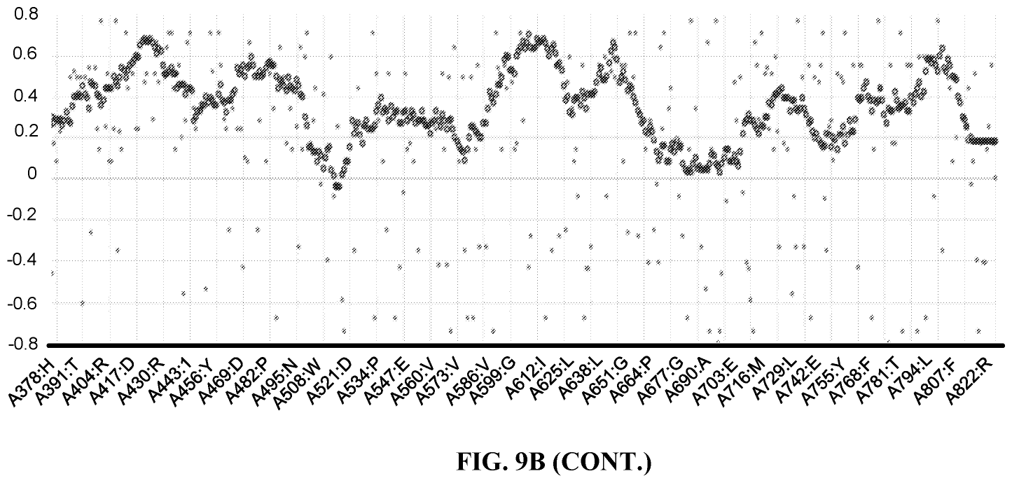

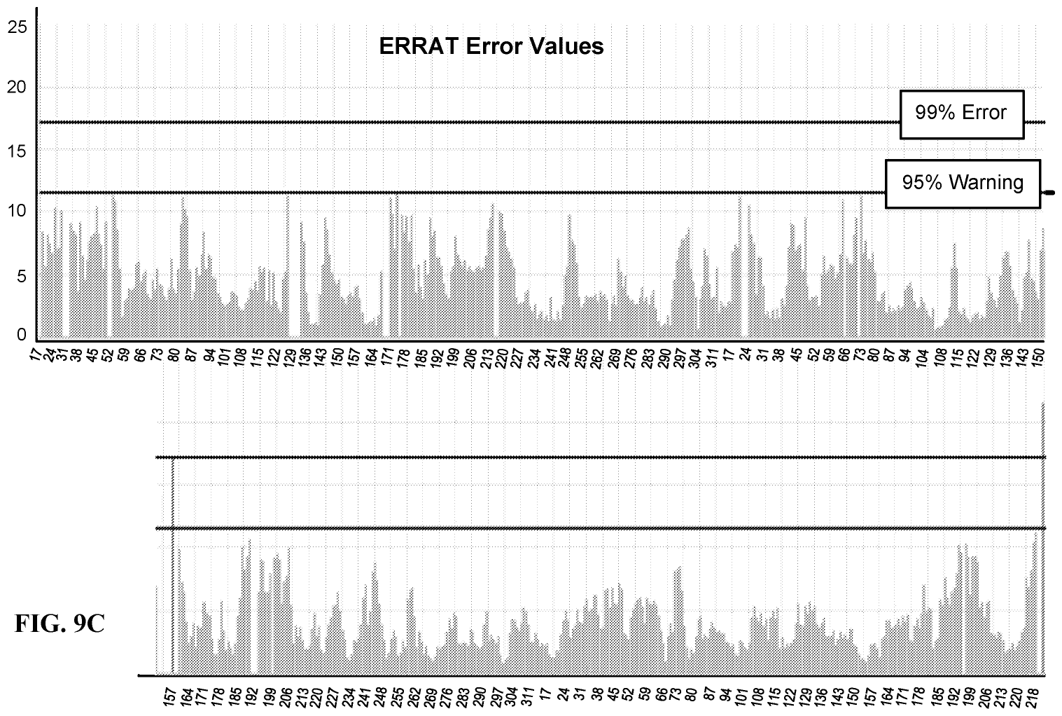

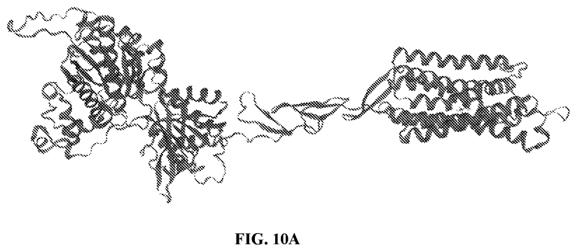

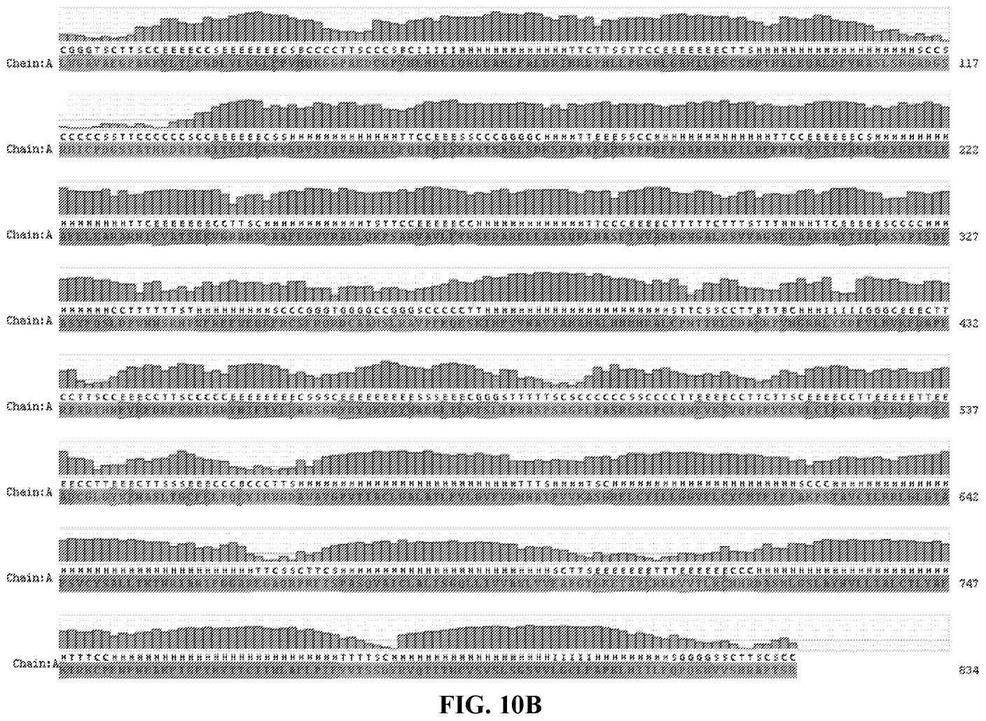

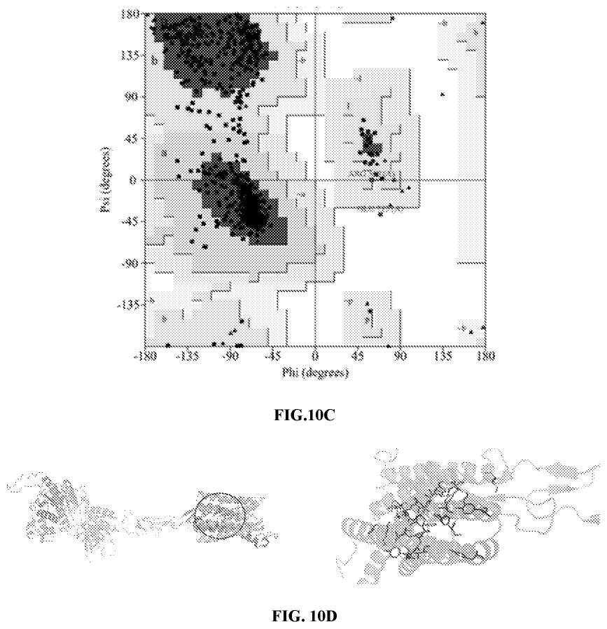

FIG. 7A shows synthetic scheme for radiolabeling of compound 13. [¹¹C]13 was prepared via the one-step O-methylation of phenol 24 (1.6 μmol) in anhydrous dimethylformamide (DMF, 0.35 mL) using [¹¹C]CH₃I in the presence of 0.5N NaOH (3.0 L). The reaction was carried out at 80° C. for 3 min, quenched by addition of 1.0 mL water, and purified by a semipreparative HPLC system. Noteworthy, the HPLC fractions containing [¹¹C]13 could be trapped on a C-18 cartridge and released via 0.6-1.0 mL ethanol with more than 95% recovery rate (n=5). In the previous radiotracer synthesis, such as [¹¹C]QCA (7), the product was enriched by removing the HPLC solvents under reduced pressure. At the end of synthesis (EOS=45 min), [¹¹C]13 was obtained with a radiochemical yield of 42±5% (n=5, non-decay corrected) calculated from starting [¹¹C]CO₂, excellent chemical and radiochemical purities (>99%), and a high molar activity (Aₘ) of 212±76 GBq/μmol (n=5). As a representative 3,4-dihydro-2H-pyrano[2,3-b]pyridine NAM tracer, [¹¹C]13 was characterized using in vivo PET imaging studies in rats and a non-human primate.

FIG. 7B shows synthetic scheme leading to compound [¹⁸F]mG2N002.

The first attempt included Ru-mediated deoxyfluorination of compound 23 using [¹⁸F]fluoride. However, under the typical radiofluorination conditions, 23 readily decomposed without forming [¹⁸F]22a.

### Radiosynthesis of S-(2-fluoro-4-[¹⁸F]fluorophenyl)-2,2-dimethyl-3,4-dihydro-2H-pyrano[2,3-b]pyridine-7-carboxamide ([¹⁸F]mG2N002)

4-(7-Cyano-2,2-dimethyl-3,4-dihydro-2H-pyrano[2,3-b]pyridin-5-yl)-3-fluorophenyl trifluoromethanesulfonate (27). To a solution of 23 (0.14 g, 0.47 mmol) and pyridine (0.22 mL) in dichloromethane (1.2 mL) was added trifluoromethanesulfonic anhydride solution (1.0 M in dichloromethane, 0.705 mL, 0.705 mmol) at 0° C. drop wisely. The reaction was then stirred at room temperature overnight. After the reaction was completed, it was quenched with saturated NaHCO₃ aqueous solution (5 mL) and extracted with ethyl acetate (3×10 mL). The combined organic solvent was dried over MgSO₄ and evaporated under reduced pressure. The resulting residue was purified by silica flash column chromatography to give the product as a white solid (0.175 g, 86.6% yield). LC-MS [M+H]⁺=431.1.

5-(2-Fluoro-4-(4,4,5,5-tetramethyl-1,3,2-dioxaborolan-2-yl)phenyl)-2,2-dimethyl-3,4-dihydro-2H-pyrano[2,3-b]pyridine-7-carbonitrile (25). To a solution of 27 (125 mg, 0.29 mmol), in 1,4-dioxane (6.0 mL) was added B₂Pin₂ (73.7 mg, 0.29 mmol), Pd(dppf)Cl₂ (21.25 mg, 0.029 mmol), and KOAc (85.5 mg, 0.87 mmol). The mixture was stirred at 80° C. for 16 h. After that the reaction was cooled to room temperature and the volatiles were removed under vacuum. The residue was purified by silica flash column chromatography to give the product as a waxy yellow solid (21.0 mg, 18.0% yield). LC-MS [M+H]⁺=409.4.

[¹⁸F]mG2N002 was synthesized via the one-pot, two-step reaction. Radiofluorination of [¹⁸F]22a was performed in the GE TRACERLab™ FX_{FN} platform following the same procedure as described for [¹⁸F]JNJ-46356479 (Yuan, G. et al. RSC. Adv. 2020, 10, 25223-25227.) Briefly, in a GE PETtrace 16.5 MeV cyclotron (GE Healthcare, Waukesha, WI, USA), the no carrier added [¹⁸F]F⁻ was produced via the ¹⁸O(p, n)¹⁸F reaction by irradiating ¹⁸O-enriched water (Isoflex Isotope, San Francisco, CA). The [¹⁸F]F⁻ aqueous solution was passed through a QMA Sep-Pak Cartridge (Sep-Pak plus light, Waters, Milford, MA) and then released by a solution of tetraethylammonium bicarbonate (TEAB, 2.7 mg, 14.1 μmol) in acetonitrile/water (0.7 mL/0.3 mL) into the reactor. The anhydrous [¹⁸F]F⁻ was obtained via azeotropic drying that was performed at 80° C. for 10 min and then 100° C. for 3 min with addition of another 1.0 mL anhydrous acetonitrile (1 mL) in the FX_{FN} platform. After the reactor was cooled to 50° C., 0.4 mL n-BuOH was added followed by a solution of 25 (6.2 μmol) and [Cu(OTf)₂Py₄](13.3 μmol) in dimethylacetamide (DMA, 0.8 mL). The reaction was heated at 110° C. for 10 min to yield [¹⁸F]22a. After that, the reaction was cooled to 50° C. and quenched with 4.0 mL of water. A solution of K₂CO₃ (12 mg) in 30% H₂O₂ (0.6 mL) was then added to this mixture and it was stirred for 10 min at room temperature to hydrolyze [¹⁸F]22a to [¹⁸F]mG2N002.

[¹⁸F]mG2N002 was isolated from the reaction mixture via a semi-preparative HPLC system equipped with an Xbridge BEH C₁₈ OBD column (130 Å, 5 μm, 10×250 mm). The purification was performed with an eluent of acetonitrile: 0.1% triethylamine solution [55:45 (v/v)] at a flow rate of 5 mL/min. The fraction containing [¹⁸F]mG2N002 was collected at a retention time of 8.5 min. The fraction was then diluted with 25 mL high purity water and trapped onto a C18 cartridge (light Sep-Pak, Waters, Milford, MA). The C18 cartridge was further washed with 10 mL water before it was treated with 0.6 mL ethanol and 5.4 mL saline to release and formulate [¹⁸F]mG2N002 into 10% ethanolic saline solution for animal injection. The radiochemical identity, molar activity (Aₘ), and purity of the injected radioligands were determined by the analytical radio-HPLC system (Waters 4000) using an XBridge analytical column (C18, 3.5 μm, 4.6×150 mm) eluted with acetonitrile: 0.1% triethylamine solution [60:40 (v/v)] at 1 mL/min and a UV wavelength of 254 nm.

At the end of synthesis (EOS=55 min), [¹⁸F]mG2N002 was obtained with a radiochemical yield of 10±3% (n=5, non-decay corrected), a high molar activity (Aₘ) of 180±36 GBq/μmol (n=3), and an excellent radiochemical purity (>99%).

FIG. 7C shows additional synthetic strategy that could be implemented to provide compound [¹¹C]12. The first attempt was made using the palladium catalyzed cyanation of compound 21a with [¹¹C]HCN and subsequent amidation of [¹¹C]22a with hydrogen peroxide to get [¹¹C]12. Unfortunately, although the unlabeled compound 22a could be prepared from compound 21a with a 71% yield at 160° C. for 30 min under the microwave conditions, [¹¹C]22a was not obtained under the conventional heating at 160° C. for 5- or 10-min when R═Cl. Precursor 21a when R═Cl was intact at 180° C. for 20 min, indicating its insufficient reactivity for such radiosynthesis. In addition, the significantly changed stoichiometry between the precursor 21a and the cyanide source might contribute to this failure when R═Cl considering [¹¹C]CN—was in the nano- or pico-molar scale. However, replacement of the aryl chloride in compound 21a with aryl bromide or iodide (R═Br or I) could allow the incorporation of [¹¹C]CN group due to the enhanced reactivity.

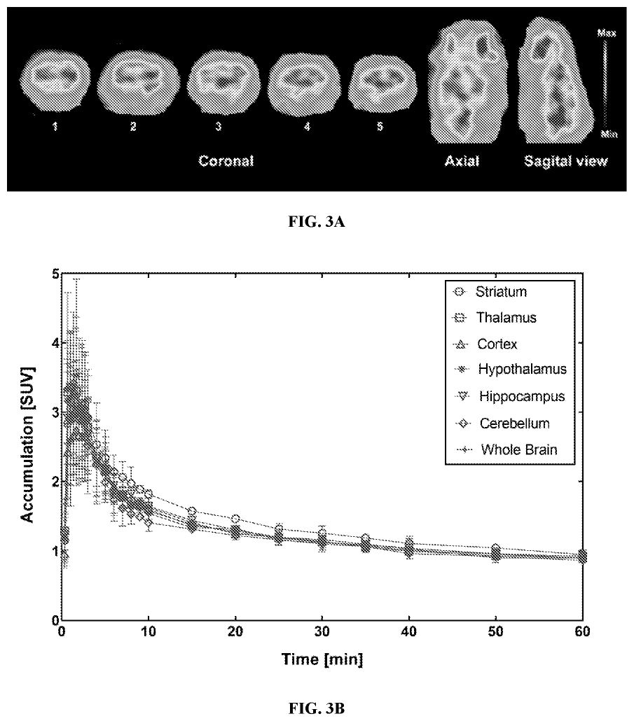

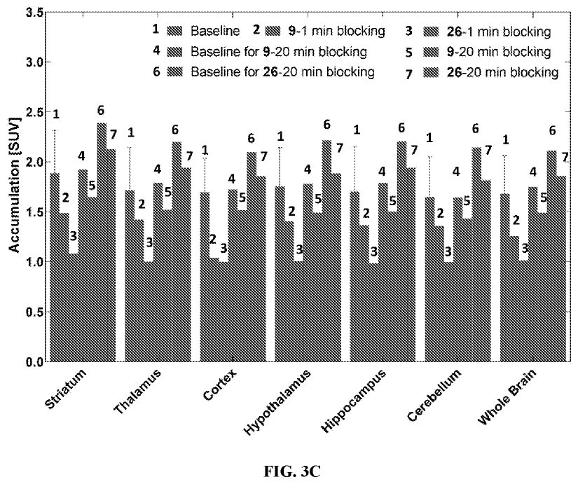

PET Imaging Studies in Rats (compound [¹¹C]13). Preliminary PET imaging studies of [¹¹C]13 were carried out in Sprague Dawley rats. Representative TACs and summed PET images at time interval of 1-30 min are shown in FIG. 3. [¹¹C]13 showed excellent brain permeability with a maximum SUV value of 3.6 at 3 min in striatum, which was higher than that of [¹¹C]MG-1904 (10, SUVₘₐₓ=1.7) and [¹¹C]MG2-1812 (11, SUVmax=1.2). [¹¹C]13 had a satisfactory tracer kinetics with most of the radioactivity washed out at 30 min (SUV₃ₘᵢₙ/SUV₃₀ₘᵢₙ=2.7). Accumulation of [¹¹C]13 was high at the mGluR2-rich regions of striatum, thalamus, cortex, hypothalamus, hippocampus, and cerebellum. [¹¹C]13 showed improved brain heterogeneity compared to those of [¹¹C]MG-1904 (10) and [¹¹C]MG2-1812 (11).

The binding specificity of [¹¹C]13 was examined by pretreatment studies with the selective mGluR2 NAM VU6001966 (9) and the potent group II NAM MNI-137 (26, IC₅₀=8.3 nM). Pretreatment with both compounds were investigated using two different time points, namely, 1 min and 20 min before radioactivity. Pretreatment with 9 (0.5 mg/kg, iv.) 1 min before tracer injection decreased the radioactivity accumulations by 22.4±7.3% across these regions of interest (ROIs) with the cortex having the highest decrease of 38.5% and thalamus the least decrease of 17.1%. However, the blocking effect significantly decreased when this agent was administered 20 min before radioactivity, where the total average decrease was 14.5 f 1.5%. Administration of 26 (0.2 mg/kg, iv.) 1 min before [¹¹C]13 induced a higher radioactivity decrease among these ROIs by 41.7±1.1% with the hypothalamus having the highest decrease of 42.6% and the cerebellum the least decrease of 39.6%. When compound 26 was administered 20 min before [¹¹C]13, the blocking effect significantly diminished with an average decrease of 12.7±1.7%. The highest decrease was observed in the cerebellum (15.2%) and the lowest decrease was seen in the striatum (11.1%). Therefore, both compounds 9 and 26 showed a similar blocking pattern where the highest blocking effect occurred when these blocking agents were administered 1 min before radioactivity, whereas this blocking effect diminished with extended time gap of 20 min. It is hypothesized the blocking agents and/or their induced pharmacological effects might wash out overtime. Altogether, [¹¹C]13 demonstrated a moderate-to-high level of specific binding toward mGluR2 in rat studies.

PET imaging study of [¹F]mG2N002 in Alzheimer's mouse model.

Animal compliance, PET imaging protocol, and image analysis are the same as the ones described in Yuan, G. et al. J. Med. Chem. 2022, 65(3), 2593-2609 under the sections of “Animal Procedures” and “PET imaging studies in rats”, except the animal model (i.e., mouse) and the amount of injected radioactivity (i.e., 120 μCi in 0.05 ml for each mouse). Animal model: 10-month-old male and female 3xTg-AD and control mice (129/C57BL6). Results: Overall, [¹⁸F]mG2N002 had excellent brain permeability with PET images showing its superior brain heterogeneity and consistent radioactivity distribution at brain regions that are enriched with mGluR2. Overall, [¹F]mG2N002 had excellent brain permeability with PET images showing its superior brain heterogeneity and consistent radioactivity distribution at brain regions that are enriched with mGluR2.

PET Imaging Studies in A Non-human Primate. To further characterize [¹¹C]13 as an imaging tool for mGluR2, the PET imaging studies were performed in a cynomolgus monkey. Brain imaging in non-human primate (NHPs) is a pivotal translational approach to study the etiology of human neuropsychiatric diseases, such as schizophrenia and drug addiction. Herein, [¹¹C]13 was characterized for its in vivo metabolism in arterial whole-blood (WB) and plasma (PL) as well as for its binding in brain tissues by using kinetic modeling techniques. This effort facilitate the application of [¹¹C]13 in humans.

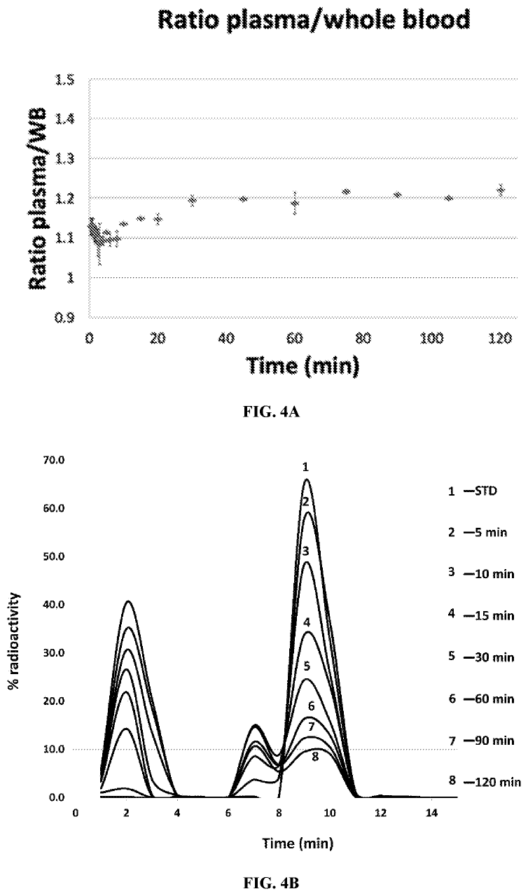

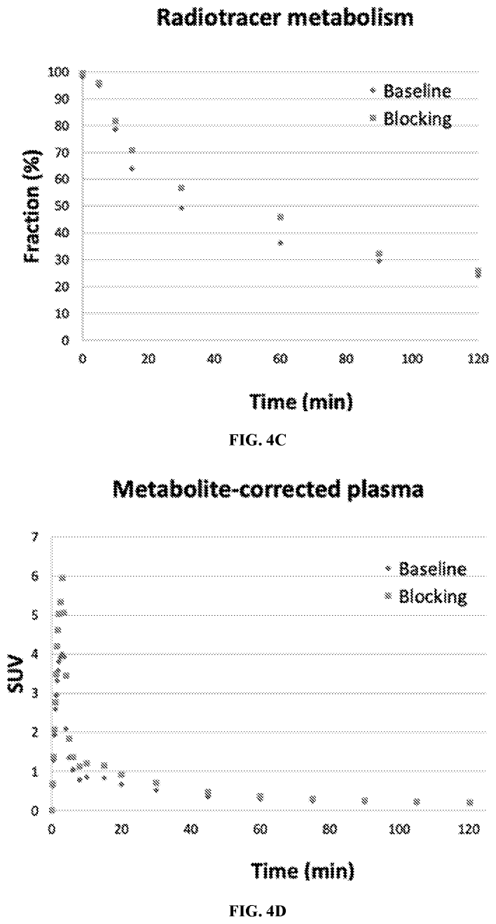

FIG. 4 shows analyses of [¹¹C]13 in arterial blood during the experimental PET imaging studies under the baseline and blocking conditions. The PL/WB ratio was similar in both studies and reached a plateau after 30 min of [¹¹C]13 injection with a mean value of 1.19±0.013. FIG. 4B shows a representative radiometabolite analysis of [¹¹C]13 with selected plasma samples. It revealed the presence of a highly polar metabolite with a retention time (t_{R}) of 2.0 min, which was likely the by-product of the [¹¹C]CH₃⁻ cleaved from the phenolic methyl ether of [¹¹C]13. Besides, there is another polar metabolite near [¹¹C]13 with a t_{R} of 6 min, the structure of which was difficult to identify due to its extremely low amount as a tracer and its absence in neither the in vitro plasma nor microsome stability assays. The top possible sites for the metabolism of 13 were predicted via SMARTCyp, where the phenolic methyl ether was ranked as the first labile group followed by the C3-C4 bond on 3,4-dihydro-2H-pyran and the pyridine nitrogen (see experimental details). Measurement of the percent parent (% PP) in plasma revealed a moderate metabolism stability with 53±5.3% of radioactivity attributable to unmetabolized [¹¹C]13 at 30 min and 24.8±1.23% at 120 min (FIG. 4C). The individual metabolite-corrected [¹¹C]13 SUV time courses in plasma is shown in FIG. 4D. The plasma-free fraction (fp) of [¹¹C]13 at baseline condition (0.131±0.006) was slightly higher than that in the blocking study (0.099±0.011). The parent fraction curve of [¹¹C]13 fitted well with a Hill function.

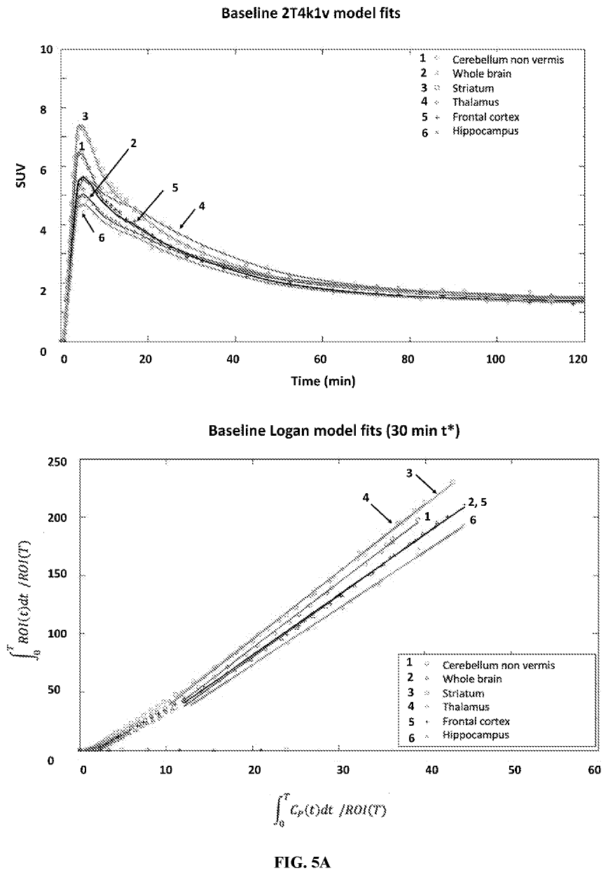

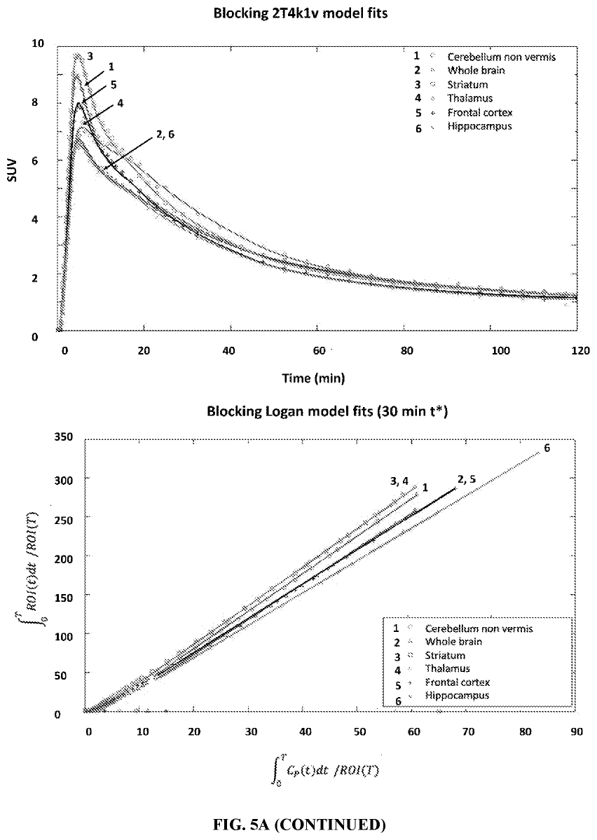

As shown in FIG. 5A, [¹¹C]13 readily crossed the BBB and peaked at 4 min after tracer injection with a SUV value of 7.5 in the striatum in baseline condition. Selected brain regions of striatum, cerebellum non vermis, thalamus, frontal cortex and hippocampus are shown. Pharmacokinetic modeling of [¹¹C]13 was best described by a reversible 2-tissue compartment model (2T4k1v) with a fixed vascular contribution v included. According to the Akaike information criteria (AIC), the 2T4k1v model provided stable regional total volume of distribution (V_{T}) estimates, which symbolize the equilibrium ratio of [¹¹C]13 in tissue to plasma as shown in FIG. 5A (left). Meanwhile, the Logan plots linearized well with t*30 min and resulted in V_{T} estimates that were well correlated with those derived from the 2T model despite an underestimation (mean difference equals to 20±6%) as depicted in FIG. 5A (right). The high K_{I} values (0.7 mL/min/cc) based on the 2T4k1v model indicated high brain penetration. In the pretreatment study, compound 9 was administered 20 min before tracer injection at a dose of 1.0 mg/kg (iv.) considering the species and metabolic rate differences between rodents and NHPs. The V_{T} estimates decreased in all ROIs over the entire acquisition. Representative Logan V_{T} estimates obtained when using 120 min and t* of 30 min are shown in FIG. 5B-C, where the decrease of V_{T} estimates ranges from 16.8% in the cerebellum gray to 3.2% in the occipital gyrus with the average decrease in the whole brain as 14.1%.

### Conclusion

Radiolabeled compounds were synthesized and characterized, including 3,4-dihydro-2H-pyrano[2,3-b]pyridine NAM 13 and compound [¹⁸F]mG2N002 as a PET imaging ligands for mGluR2. Both compounds, including compound 13, have a potent negative allosteric modulatory activity and suitable physiochemical properties as a PET imaging candidate. Radiolabeling of compound 13 was achieved via the O-methylation of phenol 24 using [¹¹C]CH₃I with a high radiochemical yield and a high molar activity. Preliminary PET imaging studies in rats confirmed the superior brain heterogeneity of [¹¹C]13, particularly in striatum and cortex, as well as its favorable binding specificity and binding kinetics. Subsequent characterization of [¹¹C]13 in a non-human primate confirmed its capability of generating high-contrast images to map the biodistribution of mGluR2 in monkey brain. Using the 2-tissue compartment model, the accumulation of [¹¹C]13 was quantified in mGluR2-enriched brain regions, where the regional total volume of distributions (V_{T}) was selectively reduced following the pretreatment of VU6001966 (9). Therefore, the experimental imaging studies conducted in two different species provided similar results in revealing the biological function of [¹¹C]13. The experimental data provides credible evidence that [¹¹C]13 and [¹⁸F]mG2N002 are PET imaging ligands for mGluR2.

### Experimental Details

All reagents and starting materials were obtained from the commercial sources including Sigma-Aldrich (St. Louis, MO), Thermo Fisher Scientific, Combi-Blocks (San Diego, CA), Ambeed (Arlington Hts, IL), and used as received. The commercially available compounds VU6001966 (9) and MNI-137 (26) were purchased from Tocris Bioscience (Minneapolis, MN). Silica gel flash column chromatography was performed using silica gel, particle size 60 A, 230-400 mesh (Supelco). Microwave reactions were carried out in a CEM Discover microwave synthesizer. ¹H and ¹³C nuclear magnetic resonance (NMR) spectra were collected with a JEOL 500 MHz spectrometer using tetramethylsilane (TMS) as an internal standard. All chemical shifts (δ) are assigned as parts in per million (ppm) downfield from TMS. Signals are described as s (singlet), d (doublet), t (triplet), q (quartet), or m (multiplet). Coupling constants (J) are quoted in hertz. Liquid chromatography-mass spectrometry (LCMS) was used to determine the mass and purity of all compounds (≥95%). The LCMS is equipped with a 1200 series HPLC system (Agilent Technologies, Canada), a multi-wavelength UV detector, a model 6310 ion trap mass spectrometer (Santa Clara, CA), and an analytical column (Agilent Eclipse C8, 150 mm×4.6 mm, 5 μm). High-Resolution Mass Spectrometry (HRMS) was obtained from the Harvard Center for Mass Spectrometry at the Harvard University, Cambridge, using electrospray ionization (ESI) technique (Thermo_q-Exactive_Plus_I Mass Spectrometer).

Chemistry, Methyl (E)-3-(2,6-dichloro-4-iodopyridin-3-yl)acrylate (16). To a 15 solution of 2,6-dichloro-4-iodonicotinaldehyde (14, 5.0 g, 16.56 mmol) in anhydrous tetrahydrofuran (105 mL) was added methyl 2-(triphenylphosphoranylidene)acetate (15, 8.31 g, 24.84 mmol) under nitrogen. The mixture was stirred at 80° C. for 2h. After the reaction was completed, the solvent was evaporated under vacuum and the residue was purified by silica flash column chromatography to give the product as pale-yellow solid (14.16 mmol, 5.07 g, 85.5% yield). ¹H NMR (500 MHz, CD₃OD): δ 7.84 (s, 1H), 7.54 (d, J=16.3 Hz, 1H), 6.44 (d, J=16.3 Hz, 1H), 3.84 (s, 3H). ¹³C NMR (125 MHz, CDCl₃): δ 165.8, 149.7, 147.6, 141.3, 133.7, 133.1, 128.1, 112.4, 52.33. HRMS (ESI⁺) for C₉H₇Cl₂INO₂⁺ [M+H]⁺ requires m/z=357.8893, found 357.8891.

Methyl 3-(2,6-dichloro-4-iodopyridin-3-yl)propanoate (17). To a solution of 16 (5.5 g, 15.36 mmol) in anhydrous tetrahydrofuran/tert-Butanol (21 mL/21 mL) was added the RhCl(PPh₃)₃ (2.82 g, 3.05 mmol). The mixture was stirred at room temperature under 42 psi H₂ for 48 h. After the reaction was completed. The solvent was removed under vacuum and the residue was purified by silica flash column chromatography to give the product as white solid (3.14 g, 56.8% yield). ¹H NMR (500 MHz, CDCl₃): δ 7.74 (s, 1H), 3.72 (s, 3H), 3.27 (t, J=8.4 Hz, 2H), 2.56 (t, J=8.4 Hz, 2H). ¹³C NMR (125 MHz, CDCl₃): δ 172.1, 148.6, 148.4, 136.4, 133.7, 114.0, 52.13, 33.1, 31.6. HRMS (ESI⁺) for C₉H₉Cl₂INO₂⁺ [M+H]⁺ requires m/z=359.9050, found 359.9049.

Methyl 3-(2,6-dichloro-4-(2,4-difluorophenyl)pyridin-3-yl)propanoate (19a). To a solution of 17 (0.5 g, 1.39 mmol) in 1,4-dioxane/water (3.0 mL/0.6 ML) was added (2,4-difluorophenyl)boronic acid (18a, 0.24 g, 1.53 mmol), Pd(dppf)Cl₂ (0.10 g, 0.139 mmol), and NaHCO₃ (0.234 g, 2.78 mmol). The mixture was stirred at 100° C. for 3 h. The solvent was removed under vacuum and the residue was purified by silica flash column chromatography to give the product as a yellow oil (0.23 g, 47.8% yield). ¹H NMR (500 MHz, CDCl₃): δ 7.17 (dd, J=6.7, 14.8 Hz, 1H), 7.12 (s, 1H), 7.01 (t, J=8.2 Hz, 1H), 6.96 (t, J=9.1 Hz, 1H), 3.60 (s, 3H), 2.88-2.91 (m, 2H), 2.48 (t, J=7.7 Hz, 2H). ¹³C NMR (125 MHz, CDCl₃): δ 172.3, 163.6 (dd, J=11.8, 252.3 Hz), 159.0 (dd, J=12.1, 250.5 Hz), 151.2, 148.4, 148.0, 132.6, 131.3 (dd, J=4.2, 9.7 Hz), 125.0, 120.9 (d, J=12.5 Hz), 112.3 (dd, J=3.4, 21.4 Hz), 104.9 (t, J=25.4 Hz), 51.9, 32.2, 25.4. ¹⁹F NMR (470 MHz, CDCl₃): δ −106.99 (dd, J=5.9, 13.0 Hz), −109.20 (dd, J=7.6, 16.1 Hz). HRMS (ESI⁺) for C₁₅H₁₂Cl₂F₂NO₂⁺ [M+H]⁺ requires m/z=346.0208, found 346.0208.

Methyl 3-(2,6-dichloro-4-(2-fluoro-4-methoxyphenyl)pyridin-3-yl)propanoate (19b). The procedure described for compound 19a was applied to (2-fluoro-4-methoxyphenyl)boronic acid (18b) to give compound 19b as a waxy pale-yellow solid (0.383 g, 77.0% yield). ¹H NMR (500 MHz, CDCl₃): δ 7.11 (s, 1H), 7.06 (t, J=8.5 Hz, 1H), 6.78 (dd, J=2.2, 8.5 Hz, 1H), 6.71 (dd, J=2.3, 11.5 Hz, 1H), 3.84 (s, 3H), 3.60 (s, 3H), 2.92 (t, J=8.0 Hz, 2H), 2.47 (t, J=8.2 Hz, 2H). ¹³C NMR (125 MHz, CDCl₃): δ 172.5, 161.9 (d, J=10.8 Hz), 159.5 (d, J=246.9 Hz), 151.0, 149.4, 147.8, 132.8, 130.8 (d, J=4.4 Hz), 125.2, 116.7 (d, J=16.4 Hz), 110.8, 102.1 (d, J=25.3 Hz), 55.8, 51.9, 32.2, 25.5. ¹⁹F NMR (470 MHz, CDCl₃): δ −111.6 (t, J=9.0 Hz). HRMS (ESI⁺) for C₁₆H₁₅Cl₂FNO₃⁺ [M+H]⁺ requires m/z=358.0408, found 358.0408.

Methyl 3-(4-(4-(benzyloxy)-2-fluorophenyl)-2,6-dichloropyridin-3-yl)propanoate (19c). The procedure described for compound 19a was applied to (4-(benzyloxy)-2-fluorophenyl)boronic acid (18c) to give compound 19c as a colorless oil (0.46 g, 84.0%). ¹H NMR (500 MHz, CDCl₃): δ 7.40-7.44 (m, 4H), 7.34-7.37 (m, 1H), 7.12 (s, 1H), 7.07 (t, J=8.5 Hz, 1H), 6.86 (dd, J=2.4, 8.5 Hz, 1H), 6.79 (dd, J=2.4, 11.5 Hz, 1H), 5.09 (s, 2H), 3.60 (s, 3H), 2.93 (t, J=8.2 Hz, 2H), 2.48 (t, J=8.2 Hz, 2H). ¹³C NMR (125 MHz, CDCl₃): δ 172.5, 161.0 (d, J=10.8 Hz), 159.4 (d, J=247.3 Hz), 151.0, 149.4, 147.8, 136.0, 132.8, 130.8, 128.9, 128.5, 127.7 (m), 125.2 (m), 117.0 (d, J=16.6 Hz), 111.5, 103.1 (d, J=25.2 Hz), 70.6, 51.9, 32.3, 25.5. ¹⁹F NMR (470 MHz, CDCl₃): δ −111.4 (t, J=10.0 Hz). HRMS (ESI⁺) for C₂₂H₁₉Cl₂FNO₃⁺ [M+H]⁺ requires m/z=434.0721, found 434.0722.

4-(2,6-dichloro-4-(2,4-difluorophenyl)pyridin-3-yl)-2-methylbutan-2-ol (20a). To a solution of 19a (0.23 g, 0.66 mmol) in anhydrous tetrahydrofuran (6.3 mL) was added methylmagnesium bromide (3.0 M in diethyl ether, 1.33 mL, 4.0 mmol) dropwise at 0° C. under nitrogen. The mixture was stirred at 0° C. for 1 h. After the reaction was completed, the mixture was quenched with saturated aqueous NH₄Cl solution (30 mL) and extracted with ethyl acetate (20 mL×3). The combined organic layers were dried over anhydrous MgSO₄. The solvent was removed under reduced pressure and the residue was purified by silica flash column chromatography to give the product as a colorless oil (0.22 g, 95.6% yield). ¹H NMR (500 MHz, CDCl₃): δ 7.17 (td, J=6.3, 8.3 Hz, 1H), 7.10 (s, 1H), 6.99 (td, J=2.1, 7.9 Hz, 1H), 6.95 (td, J=2.4, 9.3 Hz, 1H), 2.63 (m, 2H), 1.53 (m, 2H), 1.08 (s, 6H), 1.03 (s, 1H). ¹³C NMR (125 MHz, CDCl₃): δ 163.5 (dd, J=11.6, 252.1 Hz), 159.1 (dd, J=11.9, 250.2 Hz), 151.0, 148.1, 147.4, 134.6, 131.3 (dd, J=4.3, 9.6 Hz), 124.8, 121.1 (dd, J=3.8, 16.6 Hz), 112.0 (dd, J=3.4, 21.3 Hz), 104.7 (t, J=25.5 Hz), 70.5, 42.0, 28.8, 25.4. ¹⁹F NMR (470 MHz, CDCl₃): δ −107.27 (m), −109.00 (dd, J=8.2, 16.2 Hz). HRMS (ESI⁺) for C₁₆H₁₆Cl₂F₂NO⁺ [M+H]⁺ requires m/z=346.0572, found 346.0570.

4-(2,6-dichloro-4-(2-fluoro-4-methoxyphenyl)pyridin-3-yl)-2-methylbutan-2-ol (20b). The procedure described for compound 20a was applied to 19b to give compound 20b as a colorless oil (0.28 g, 95.2% yield). ¹H NMR (500 MHz, CDCl₃): δ 7.10 (s, 1H), 7.07 (t, J=8.5 Hz, 1H), 6.77 (dd, J=2.4, 8.5 Hz, 1H), 6.71 (dd, J=2.4, 11.5 Hz, 1H), 3.83 (s, 3H), 2.67 (t, J=8.2 Hz, 2H), 1.55 (t, J=8.2 Hz, 2H), 1.08 (s, 6H), 1.02 (s, 1H). ¹³C NMR (125 MHz, CDCl₃): δ 161.8 (d, J=10.7 Hz), 159.5 (d, J=247.2 Hz), 150.8, 149.1, 147.2, 134.8, 130.8 (d, J=4.1 Hz), 125.1, 117.0 (d, J=16.8 Hz), 110.5, 102.0 (d, J=25.2 Hz), 70.7, 55.8, 42.1, 28.8, 25.4. ¹⁹F NMR (470 MHz, CDCl₃): δ −111.4 (t, J=10.1 Hz). HRMS (ESI⁺) for C₁₇H₁₉Cl₂FNO₂⁺ [M+H]⁺ requires m/z=358.0771, found 358.0771.

4-(4-(4-(benzyloxy)-2-fluorophenyl)-2,6-dichloropyridin-3-yl)-2-methylbutan-2-ol (20c). The procedure described for compound 20a was applied to 19c to give compound 20c as a pale-yellow oil (0.37 g, 92.5%). ¹H NMR (500 MHz, CDCl₃): δ 7.38-7.43 (m, 4H), 7.33-7.36 (m, 1H), 7.10 (s, 1H), 7.07 (t, J=8.5 Hz, 1H), 6.85 (dd, J=2.4, 8.5 Hz, 1H), 6.79 (dd, J=2.4, 11.4 Hz, 1H), 5.09 (s, 2H), 2.64-2.68 (m, 2H), 1.51-1.54 (m, 2H), 1.07 (s, 6H), 1.01 (s, 1H). ¹³C NMR (125 MHz, CDCl₃): δ 160.8 (d, J=10.8 Hz), 159.4 (d, J=247.2 Hz), 150.8, 149.0, 147.2, 136.0, 134.8, 130.8, 128.8 (m), 128.4 (m), 127.6 (m), 125.2 (m), 117.3 (d, J=16.8 Hz), 111.3, 103.0 (d, J=25.4 Hz), 70.6, 70.5, 42.1, 28.7, 25.4. ¹⁹F NMR (470 MHz, CDCl₃): δ −111.3 (t, J=10.0 Hz). HRMS (ESI⁺) for C₂₃H₂₃Cl₂FNO₂⁺ [M+H]⁺ requires m/z=434.1084, found 434.1086.

7-chloro-S-(2,4-difluorophenyl)-2,2-dimethyl-3,4-dihydro-2H-pyrano[2,3-b]pyridine (21a). To a solution of 20a (0.22 g, 0.64 mmol) in N,N-dimethylacetamide (10.0 mL) was added cesium carbonate (0.417 g, 1.28 mmol). The mixture was stirred at 120° C. overnight. The mixture was washed with water (30 mL) and extracted with ethyl acetate (20 mL×3). The combined organic layers were dried over anhydrous MgSO₄. The solvent was removed under reduced pressure and the residue was purified by silica flash column chromatography to give the product as a pale-yellow solid (0.068 g, 34.3% yield). ¹H NMR (500 MHz, CDCl₃): δ 7.20 (td, J=6.3, 8.4 Hz, 1H), 6.97 (ddd, J=1.2, 2.5, 8.0 Hz, 1H), 6.91 (ddd, J=2.5, 8.9, 9.8 Hz, 1H), 6.79 (s, 1H), 2.50 (m, 2H), 1.75 (t, J=6.7 Hz, 2H), 1.41 (s, 6H). ¹³C NMR (125 MHz, CDCl₃): δ 163.3 (dd, J=11.8, 251.1 Hz), 160.4, 159.3 (dd, J=11.9, 238.6 Hz), 147.4, 147.3, 131.4 (dd, J=4.8, 9.6 Hz), 121.4 (d, J=20.2 Hz), 117.8, 113.6, 111.9 (dd, J=3.5, 21.6 Hz), 104.5 (t, J=25.7 Hz), 32.0, 29.8, 27.0, 20.1, 20.0. ¹⁹F NMR (470 MHz, CDCl₃): δ −108.44 (m), −109.32 (m). HRMS (ESI⁺) for C₁₆H₁₅ClF₂NO⁺ [M+H]⁺ requires m/z=310.0805, found 310.0807.

7-chloro-S-(2-fluoro-4-methoxyphenyl)-2,2-dimethyl-3,4-dihydro-2H-pyrano[2,3-b]pyridine (21b). The procedure described for compound 21a was applied to 20b to give compound 21b as a yellow oil (0.13 g, 48.1% yield). ¹H NMR (500 MHz, CDCl₃): δ 7.09-7.13 (m, 1H), 6.78 (s, 1H), 6.75-6.79 (m, 1H), 6.69 (d, J=11.7 Hz, 1H), 3.83 (s, 3H), 2.52-2.54 (m, 2H), 1.72-1.74 (m, 2H), 1.39 (s, 6H). ¹³C NMR (125 MHz, CDCl₃): δ 161.6 (d, J=10.9 Hz), 160.3, 159.8 (d, J=247.7 Hz), 148.4, 147.0, 131.0 (d, J=5.0 Hz), 118.0, 117.4 (d, J=16.7 Hz), 113.7, 110.4, 102.0 (d, J=25.6 Hz), 77.2, 55.8, 32.1, 27.0, 20.2, 20.1. ¹⁹F NMR (470 MHz, CDCl₃): δ −111.4. HRMS (ESI⁺) for C₁₇H₁₈ClFNO₂⁺ [M+H]⁺ requires m/z=322.1005, found 322.1007.

5-(4-(benzyloxy)-2-fluorophenyl)-7-chloro-2,2-dimethyl-3,4-dihydro-2H-pyrano[2,3-b]pyridine (21c). The procedure described for compound 21a was applied to 20c to give compound 21c as a colorless oil (0.13 g, 40.4%). ¹H NMR (500 MHz, CDCl₃): δ 7.39-7.44 (m, 4H), 7.34-7.37 (m, 1H), 7.12 (t, J=8.5 Hz, 1H), 6.84 (dd, J=2.4, 8.5 Hz), 6.79 (s, 1H), 6.77 (dd, J=2.4, 11.7 Hz, 1H), 5.09 (s, 2H), 2.54 (t, J=6.4 Hz, 2H), 1.74 (t, J=6.7 Hz, 2H), 1.41 (s, 6H). ¹³C NMR (125 MHz, CDCl₃): δ 160.7 (d, J=6.0 Hz), 160.3, 159.7 (d, J=231.0 Hz), 148.3, 147.0, 136.1, 131.1 (m), 128.8 (m), 128.5 (m), 127.6 (m), 118.0, 117.7 (d, J=16.7 Hz), 113.7, 111.2, 102.9 (d, J=26.6 Hz), 77.2, 70.6, 32.1, 27.1, 20.1. ¹⁹F NMR (470 MHz, CDCl₃): δ −111.2 (t, J=9.2 Hz). HRMS (ESI⁺) for C₂₃H₂₂ClFNO₂⁺ [M+H]⁺ requires m/z=398.1318, found 398.1316.

5-(2,4-difluorophenyl)-2,2-dimethyl-3,4-dihydro-2H-pyrano[2,3-b]pyridine-7-carbonitrile (22a). To a solution of 21a (30.0 mg, 0.097 mmol) in dimethylformamide (3.0 mL) was added zinc cyanide (36.0 mg, 0.306 mmol) and tetrakis(triphenylphosphine)palladium(0) (30.0 mg, 0.026 mmol) in a microwave tube. The mixture was heated to 160° C. in a microwave synthesizer (CEM, Discover SP) for 30 min. The reaction was washed with water (20 mL) and extracted with ethyl acetate (20 mL×3). The combined organic layers were dried over anhydrous MgSO₄. The solvent was removed under reduced pressure and the residue was purified by silica flash column chromatography to give the product as a pale-yellow waxy solid (18.0 mg, 61.8% yield). ¹H NMR (500 MHz, CDCl₃): δ 7.21 (td, J=6.3, 8.4 Hz, 1H), 7.16 (s, 1H), 7.02 (td, J=2.2, 8.3 Hz, 1H), 6.95 (ddd, J=2.4, 8.8, 9.9 Hz, 1H), 2.63 (m, 2H), 1.80 (t, J=6.7 Hz, 2H), 1.44 (s, 6H). ¹³C NMR (125 MHz, CDCl₃): δ 163.6 (dd, J=11.6, 252.3 Hz), 161.5, 159.3 (dd, J=12.0, 251.0 Hz), 146.0, 131.3 (dd, J=4.6, 9.6 Hz), 129.9, 123.2, 121.0, 120.6 (d, J=16.3 Hz), 117.0, 112.3 (d, J=21.3 Hz), 104.8 (t, J=25.5 Hz), 77.9, 31.6, 27.1, 20.9, 20.8. ¹⁹F NMR (470 MHz, CDCl₃): δ −107.32 (dd, J=6.5, 13.8 Hz), −109.04 (dd, J=7.4, 17.4 Hz). HRMS (ESI⁺) for C₁₇H₁₈F₂N₂O⁺ [M+H]⁺ requires m/z=301.1147, found 301.1149.

5-(2-fluoro-4-methoxyphenyl)-2,2-dimethyl-3,4-dihydro-2H-pyrano[2,3-b]pyridine-7-carbonitrile (22b). The procedure described for compound 22a was applied to 21b to give compound 22b as a colorless waxy solid (18.0 mg, 52.3% yield). ¹H NMR (500 MHz, CDCl₃): δ 7.16 (s, 1H), 7.12 (t, J=8.5 Hz, 1H), 6.80 (dd, J=2.4, 8.5 Hz, 1H), 6.71 (dd, J=2.4, 11.8 Hz, 1H), 3.85 (s, 3H), 2.67 (t, J=6.3 Hz, 2H), 1.78 (t, J=6.6 Hz, 2H), 1.43 (s, 6H). 13C NMR (125 MHz, CDCl₃): δ 162.0 (d, J=10.9 Hz), 161.4, 159.8 (d, J=248.0 Hz), 146.9, 130.9 (d, J=4.7 Hz), 129.6, 123.6, 121.1, 117.2, 116.4 (d, J=16.4 Hz), 110.7, 102.1 (d, J=25.5 Hz), 77.7, 55.9, 31.8, 27.2, 21.0. ¹⁹F NMR (470 MHz, CDCl₃): δ −111.1 (t, J=8.8 Hz). HRMS (ESI⁺) for C₁₈H₁₈FN₂O₂⁺ [M+H]⁺ requires m/z=313.1347, found 313.1349.

5-(4-(benzyloxy)-2-fluorophenyl)-2,2-dimethyl-3,4-dihydro-2H-pyrano[2,3-b]pyridine-7-carbonitrile (22c). The procedure described for compound 22a was applied to 21c to give compound 22c was obtained as a pale-yellow solid (29.0 mg, 29.7%). ¹H NMR (500 MHz, CDCl₃): ¹H NMR (500 MHz, CDCl₃): δ 7.40-7.45 (m, 4H), 7.35-7.38 (m, 1H), 7.17 (s, 1H), 7.12 (t, J=8.5 Hz, 1H), 6.87 (dd, J=2.4, 8.5 Hz), 6.80 (dd, J=2.4, 11.8 Hz, 1H), 5.10 (s, 2H), 2.66 (t, J=6.6 Hz, 2H), 1.79 (t, J=6.7 Hz, 2H), 1.44 (s, 6H). ¹³C NMR (125 MHz, CDCl₃): δ 161.4, 161.0 (d, J=11.1 Hz), 159.8 (d, J=248.1 Hz), 146.9, 136.0, 130.9 (d, J=5.0 Hz), 129.6, 128.9, 128.5, 127.6, 123.5, 121.0, 117.2, 116.7 (d, J=16.6 Hz), 111.5, 103.1 (d, J=25.5 Hz), 77.7, 70.6, 31.8, 27.2, 21.0. ¹⁹F NMR (470 MHz, CDCl₃): δ −110.9 (t, J=9.4 Hz). HRMS (ESI⁺) for C₂₄H₂₂FN₂O₂⁺ [M+H]⁺ requires m/z=389.1660, found 389.1656.

5-(2,4-difluorophenyl)-2,2-dimethyl-3,4-dihydro-2H-pyrano[2,3-b]pyridine-7-carboxamide (12). To a solution of 22a (18.0 mg, 0.06 mmol) in acetone (2.0 mL) was added a solution of sodium percarbonate (43.8 mg, 0.29 mmol) in water (1.0 mL) dropwise. The mixture was stirred at room temperature for 1h. After the reaction was completed, the mixture was diluted with water (20 mL) and extracted with ethyl acetate (20 mL×3). The combined organic layers were dried over anhydrous MgSO₄. The solvent was removed under reduced pressure and the residue was purified by silica flash column chromatography to give the product as a white solid (11.0 mg, 57.6% yield). ¹H NMR (500 MHz, CDCl₃): δ 7.71 (s, 2H), 7.22-7.27 (m, 1H), 6.99 (td, J=2.4, 8.4 Hz, 1H), 6.92 (td, J=2.4, 9.4 Hz, 1H), 5.51 (s, 1H), 2.60-2.64 (m, 2H), 1.81 (t, J=6.7 Hz, 2H), 1.46 (s, 6H). ¹³C NMR (125 MHz, CDCl₃): δ 166.4, 163.3 (dd, J=11.9, 251.0 Hz), 159.6, 159.3 (dd, J=12.1, 250.3 Hz), 146.4, 146.3, 131.6 (dd, J=4.6, 9.2 Hz), 121.8 (dd, J=3.0, 16.4 Hz), 119.3, 117.6, 112.0 (d, J=21.5 Hz), 104.4 (t, J=25.7 Hz), 32.0, 29.8, 20.8. HRMS (ESI⁺) for C₁₇H₁₇F₂N₂O₂⁺ [M+H]⁺ requires m/z=319.1253, found 319.1253.

5-(2-fluoro-4-methoxyphenyl)-2,2-dimethyl-3,4-dihydro-2H-pyrano[2,3-b]pyridine-7-carboxamide (13). The procedure described for compound 12 was applied to 22b to give compound 13 as a white solid (25.0 mg, 78.9% yield). ¹H NMR (500 MHz, CDCl₃): δ 7.73 (s, 2H), 7.18 (t, J=8.5 Hz, 1H), 6.79 (dd, J=2.3, 8.5 Hz, 1H), 6.71 (dd, J=2.4, 11.8 Hz, 1H), 5.54 (s, 1H), 3.85 (s, 3H), 2.66-2.70 (m, 2H), 1.81 (t, J=6.7 Hz, 2H), 1.47 (s, 6H). ¹³C NMR (125 MHz, CDCl₃): δ 166.6, 161.5 (d, J=10.8 Hz), 159.6, 159.8 (d, J=247.5 Hz), 147.4, 146.0, 131.2 (d, J=5.2 Hz), 119.4, 117.9, 117.7, 110.4, 101.9 (d, J=25.7 Hz), 77.2, 55.8, 32.1, 27.2, 20.8. ¹⁹F NMR (470 MHz, CDCl₃): δ −111.3 (t, J=10.7 Hz). HRMS (ESI⁺) for C₁₈H₂₀FN₂O₃⁺ [M+H]⁺ requires m/z=331.1452, found 331.1454.

5-(2-fluoro-4-hydroxyphenyl)-2,2-dimethyl-3,4-dihydro-2H-pyrano[2,3-b]pyridine-7-carbonitrile (23). To a solution of 5-(4-(benzyloxy)-2-fluorophenyl)-2,2-dimethyl-3,4-dihydro-2H-pyrano[2,3-b]pyridine-7-carbonitrile (22c, 22.0 mg, 0.057 mmol) in ethyl acetate (1.3 mL) was added palladium on carbon (Pd/C) (10 wt. %, 3.0 mg). The mixture was stirred at room temperature for 1h under hydrogen. After the reaction was completed, Pd/C was filtered and the solvent was removed under reduced pressure. The residue was purified by silica flash column chromatography to give the product as white solid (6.0 mg, 35.5% yield). ¹H NMR (500 MHz, CDCl₃): δ 7.17 (s, 1H), 7.08 (t, J=8.3 Hz, 1H), 6.74 (dd, J=2.3, 8.3 Hz, 1H), 6.70 (dd, J=2.3, 11.1 Hz, 1H), 5.52 (s, 1H), 2.66 (t, J=6.8 Hz, 2H), 1.79 (t, J=6.7 Hz, 2H), 1.44 (s, 6H). ¹³C NMR (125 MHz, CDCl₃): δ 161.5, 159.8 (d, J=248.8 Hz), 158.2 (d, J=11.9 Hz), 147.0, 131.1 (d, J=4.8 Hz), 129.5, 123.6, 121.2, 117.1, 116.6 (d, J=16.6 Hz), 112.1, 103.8 (d, J=25.1 Hz), 77.8, 31.8, 27.1, 20.9. ¹⁹F NMR (470 MHz, CDCl₃): δ −111.2 (t, J=9.0 Hz). HRMS (ESI⁺) for C₁₇H₁₆FN₂O₂⁺ [M+H]⁺ requires m/z=299.1190, found 299.1191.

5-(2-fluoro-4-hydroxyphenyl)-2,2-dimethyl-3,4-dihydro-2H-pyrano[2,3-b]pyridine-7-carboxamide (24). To a solution of 5-(2-fluoro-4-hydroxyphenyl)-2,2-dimethyl-3,4-dihydro-2H-pyrano[2,3-b]pyridine-7-carbonitrile (23, 6.0 mg, 0.02 mmol) in acetone (0.6 mL) was slowly added sodium bicarbonate (9.5 mg, 0.06 mmol) in water (0.3 mL). The resulting mixture was stirred at room temperature for 1h. After the reaction was completed, the reaction was quenched with saturated aqueous NH₄Cl (1.0 mL) and extracted with ethyl acetate (5.0 mL×3). The combined organic layers were dried over anhydrous MgSO₄. The solvent was removed under reduced pressure and the residue was purified by silica flash column chromatography to give the product as white solid (4.5 mg, 70.8% yield). ¹H NMR (500 MHz, CDCl₃): δ 7.87 (s, 1H), 7.64 (s, 1H), 7.39 (s, 1H), 7.02 (t, J=8.4 Hz, 1H), 6.77 (dd, J=2.3, 8.4 Hz, 1H), 6.70 (dd, J=2.3, 11.4 Hz, 1H), 5.62 (s, 1H), 2.68 (m, 2H), 1.80 (t, J=6.7 Hz, 2H), 1.46 (s, 6H). ¹³C NMR (125 MHz, CDCl₃): δ 167.3, 159.8 (d, J=247.2 Hz), 159.7, 158.7 (d, J=11.9 Hz), 147.6, 145.3, 131.1 (d, J=5.0 Hz), 120.0, 118.2, 117.1 (d, J=16.6 Hz), 112.1 (d, J=1.9 Hz), 103.7 (d, J=25.1 Hz), 32.0, 27.2, 20.9. ¹F NMR (470 MHz, CDCl₃): δ −111.8 (t, J=10.3 Hz). HRMS (ESI⁺) for C₁₇H₁₈FN₂O₃⁺ [M+H]⁺ requires m/z=317.1296, found 317.1296.

Radiochemistry. S-(2-fluoro-4-[¹¹C]methoxyphenyl)-2,2-dimethyl-3,4-dihydro-2H-pyrano-[2,3-b]pyridine-7-carboxamide ([¹¹C]13). [¹¹C]CH₃I was prepared from the cyclotron-generated [¹¹C]C02, which was produced via the ¹⁴N(p,α)¹¹C reaction on nitrogen with 2.5% oxygen and 16 MeV protons (GE Healthcare, PETtrace). Briefly, [¹¹C]C02 was trapped on molecular sieves in a TRACERlab FX—CH₃I synthesizer (GE Healthcare) and reduced to [¹¹C]CH₄ in the presence of hydrogen at 350° C. The resulting [¹¹C]CH₄ passed through an oven containing I₂ to afford [¹¹C]CH₃I via a radical reaction. [¹¹C]CH₃I was then transferred under helium gas to a 5 mL V-vial containing precursor 24 (0.4±0.1 mg), an aqueous 0.5N NaOH (3 μL) and anhydrous DMF (350 μL). After the transfer was completed, the mixture was heated at 80° C. for 3 min. The reaction was then quenched by adding 1.0 mL of water and purified using a semi-preparative HPLC system equipped with a Waters XBridge C18 column (250×10 mm, 5μ), a UV detector (wavelength=254 nm) and a radioactivity detector. The product was eluted with a mobile phase of acetonitrile/water/Et₃N (50/50/0.1%) at a flow rate of 5 mL/min. The fractions containing [¹¹C]13 (t_{R}=8.6 min) were collected into a large dilution flask, which was pre-loaded with 23 mL of sterile water for injection, USP. The diluted solution was loaded onto a C18 light cartridge (Waters; pre-activated with 8 mL of EtOH followed by 16 mL of water) and the cartridge was washed with 10 mL of sterile water to remove traces of salts, residual acetonitrile and Et₃N. [¹¹C]13 was then released from the cartridge via 0.6 mL of dehydrated ethyl alcohol (USP) followed by 5.4 mL of 0.9% sodium chloride solution (USP) into a product collection vessel. The formulated [¹¹C]13 solution was filtered through a vented sterilizing filter (Millipore-GV 0.22μ, EMD Millipore) into a 10 mL vented sterile vial for injection. The synthesis time was ca. 45 min from end-of-bombardment. The chemical and radiochemical purities of [¹¹C]13 were determined by a HPLC system (UltiMate 3000) equipped with an analytical column (Waters, XBridge, C18, 3.5μ, 4.6×150 mm), a UV detector (λ=254 nm) and a radioactivity detector. The mobile phase of acetonitrile/water/Et₃N (45/55/0.1%) was used and the flow rate was 1 mL/min. The identity of [¹¹C]13 was confirmed by the co-injection with unlabeled compound 13.

Pharmacology. The negative allosteric modulatory activity was determined following a standard protocol by Eurofins Discovery. Briefly, 20 μL of 10k CHO cells/well in CP24™ were seeded into white walled, 384-well microplates and incubated at 37° C./5% C02 overnight. On day of testing, media is exchanged for 10 μL of HBSS/10 mM HEPES. Intermediate dilution of compounds was performed to generate 4× stocks in HBSS/10 mM HEPES and 5 μL of 4× sample is added to the cell plate. Cells were incubated at 37° C. for 15 minutes. Then, 5 μL of 4× Forskolin and 4×EC₈₀ of the challenge agonist glutamate were added and cells were incubated for 30 min at 37° C. The concentration of Forskolin was 15 μM and the concentration of glutamate was 8.9 μM. Assay signal was generated through incubation with 5 μL of cAMP XS+Ab reagent and 20 μL cAMP XS+ED/CL lysis cocktail for one hour followed by incubation with 20 μL cAMP XS+EA reagent for two hours at room temperature. Plates were read following signal generation with a PerkinElmer Envision™ instrument for chemiluminescent signal detection. The signal is normalized to EC₈₀ response (0%) and basal signal (100%). The NAM activity was analyzed using CBIS data analysis suite (ChemInnovation, CA). Percentage inhibition was calculated using the formula of % Inhibition=100%×(mean RLU of test sample-mean RLU of EC₈₀ control)/(mean RLU of forskolin positive control−mean RLU of EC₈₀ control). The assay was run in duplicate.

Molecular modeling. The mGluR2 receptor model was built in YASARA from 17 initial models based on the crystal structures of the human metabotropic glutamate receptor 5 (PDBID:4OO9), human metabotropic glutamate receptor 1 (PDBID:4OR2), metabotropic glutamate receptor 5 apo form (PDBID:6N52), and an mGluR2 structure (PDBID: 5KZN). The model was further validated by several structural analysis tools from SAVES containing VERIFY3D, ERRA, QMEAN, and ModFOLD (see experimental details). The key interacting residues were predicted by Partial Order Optimum Likelihood (POOL), which include the previously reported interacting residues of Phe623, Arg635, Phe643, His723, and Asn735. Compounds 12 and 13 were optimized and converted into PDB format in Avogadro 1.2 before docking. Molecular docking was performed into the model structure using Extra precision Induced Fit Docking in Glide.

Physiochemical Properties. Partition coefficient (Log D_{7.4}). The Log D_{7.4} was measured by mixing a test compound (0.1 mg) with n-octanol (1.0 mL) and PBS buffer (1.0 mL) at pH 7.4 in an Eppendorf tube. The tube was vortexed for 1 min before shaken at 37° C. overnight. The amount of the test compound in each phase was determined from the area under the peak at a wavelength of 254 nm in the HPLC system (UltiMate 3000). The compound was eluted with acetonitrile/water/Et₃N (45/55/0.1%) at a flow rate of 1.0 mL/min with a Waters XBridge C18 column (250×10 mm, 5μ). The Log D_{7.4} was calculated by Log([compound in octanol]/[compound in PBS]). The assay was repeated at least three times for each compound.

Rat plasma stability. The rat plasma stability was determined by our previously described method. Briefly, the test compound (2.5 μL, 1 mM DMSO stock solution) was mixed with an aliquot of rat serum (100 μL, Abcam, Inc.) in an Eppendorf tube. The tube was vortexed and incubated at 37° C. for 0 min and 60 min, separately, before the addition of 250 μL ice-cold acetonitrile. The resulting mixture was centrifuged at 10,000 g for 20 min and the supernatant was collected for analysis on the HPLC system (UltiMate 3000). The same analytical conditions were used as those in the Log D_{7.4} assay. The plasma stability value was expressed as (peak area at 60 min)/(peak area at 0 min)×100%. The assay was repeated at least three times for each compound. Compound 24 was used as internal standard.

Rat liver microsome stability. The rat liver microsome stability was measured by our previously described method. Briefly, 1.5 μL of 1 mM compound solution in DMSO was added to an Eppendorf tube containing 432 μL of PBS buffer. The tube was kept at 37° C. for 10 min before a 13 μL aliquot of the Sprague-Dawley rat liver microsome (Sigma-Aldrich, No. M9066) was added. The tube was vortexed before shaken at 37° C. for 5 min. The NADPH (50 μL, 10 mM in PBS solution) was added and the resulting mixture was incubated at 37° C. for 0 min and 60 min, separately, before the addition of 250 μL of ice-cold acetonitrile. The mixture was centrifuged at 10,000 g for 20 min and supernatant was collected for analysis on the HPLC system (UltiMate 3000). The same analytical conditions were employed as those in the Log D_{7.4} assay. The liver microsome stability value was expressed as (peak area at 60 min)/(peak area at 0 min)×100%. The assay was repeated at least three times for each compound. Compound 24 was used as internal standard and N-(4-chloro-3-methoxyphenyl)pyridine-2-carboxamide (ML128) was employed as positive control.

Pgp-Glo™ assay. The Pgp-Glo™ assay was performed by following our previously described method and using the manufacturer's instructions (Promega, Co. USA). Briefly, 25 μg of Pgp membrane (Promega, Cat. #V3601) was added to a 96-well plate (Thermo Lab systems, Cat. #9502887) containing untreated samples, Na₃VO₄ (100 μM), Verapamil (100 μM), and tested compounds (100 μM). The Pgp ATPase reaction was activated by adding a solution of MgATP in the assay buffer (5 mM). After a brief mixing, the 96-well plate was placed in a 37° C. incubator for 40 min. The assay was then treated with 50 μL ATP detection solution and incubated at room temperature for 20 min to develop luminescent signal. The luminescence was read on an in vivo imaging system (IVIS® Spectrum, PerkinElmer, USA). The change in luminescence relative to the Na₃VO₄ samples represents the Pgp ATPase activity with a unit of photon per second (p/s). The assay was repeated at least three times for each compound.

PET imaging studies in rats. PET imaging experiments and data analysis of [¹¹C]13 in rats were performed by our previously described methods. Briefly, the imaging studies were carried out in Triumph II Preclinical Imaging System (Trifoil Imaging, LLC, Northridge, CA). Six normal Sprague Dawley rats (male, 285-421 g) were used which resulted in eight imaging studies comprising four baseline studies, two pretreatment studies with VU6001966 (9), and two blocking experiments with MNI-137 (26). For the imaging studies, rats were anesthetized with isoflurane (1.0-1.5%) and oxygen (1-1.5 L/min) and the vital signs, such as heart rate and breathing, were monitored. The data acquisition for 60 min started from the injection of [¹¹C]13 (63.0-87.3 MBq, iv.) through the tail vein using a catheter. The blocking agent 9 (0.5 mg/kg) was dissolved in a solution of 10% ethanol and 5% Tween-80 in 85% saline (0.1 mg/mL) while 26 (0.2 mg/kg, iv.) was formulated into a solution of 10% DMSO and 5% Tween-20 in 85% PBS (0.25 mg/mL). The blocking agents were administered 1 or 20 min before the tracer injection.

After each PET acquisition, a CT scan was performed to provide anatomical information and data for attenuation correction. The list mode PET data were reconstructed to twenty-four dynamic volumetric images (9×20s, 7×1min, 6×5 min, 2×10 min) via the maximum-likelihood expectation-maximization (MLEM) algorithm with 30 iterations. The ROIs, i.e., striatum, frontal cortex, cingulate cortex, hippocampus, hypothalamus, thalamus, and cerebellum were drawn onto coronal PET slices according to the rat brain atlas. The time activity curves for these ROIs were generated by PMOD 3.2 (PMOD Technologies Ltd., Zurich, Switzerland).

PET imaging studies in a nonhuman primate. PET imaging experiments, arterial blood sampling, and data analysis of [¹¹C]13 in a cynomolgus monkey (Macaca fascicularis) (5.0 kg, female) were done by the previously described methods.

PET imaging. The PET scans were performed in a Discovery MI (GE Healthcare) PET/CT scanner. Prior to each study, the monkey was sedated with ketamine/xylazine (10/0.5 mg/kg IM) and maintained under anesthesia with a flow of isoflurane (1-2%) in oxygen. A CT scan was done before each PET acquisition to verify anatomical location and get data for attenuation correction. The PET data acquisition started immediately at the start of a 3-minute tracer infusion and lasted for 120 min. Radiotracer activity injected at baseline and blocking studies was 190.55 MBq (Aₘ=288.5 GBq/μmol) and 239.39 MBq (Aₘ=135.6 GBq/μmol). The blocking agent, 9 (1.0 mg/kg, iv.) was administered 20 min before tracer injection. After the PET scan, the acquired PET data were reconstructed via a 3D time-of-flight iterative reconstruction algorithm with 3 iterations and 34 subsets. The data were also corrected for photon attenuation and scatter, radioactive decay, system dead time, detector inhomogeneity and random coincident events. The list mode PET data were framed to fifty four dynamic volumetric images (6×10, 8×15, 6×30, 8×60, 8×120 and 18×300s) with voxel dimensions of 256×256×89 and voxel sizes of 1.17×1.17×2.8 mm³.

Arterial blood sampling and analysis. Prior to radiotracer injection, a 3-mL arterial blood sample was drawn to determine the plasma protein binding of [¹¹C]13. Briefly, the blood sample was centrifuged and an aliquot of the supernatant was spiked with [¹¹C]13 in PBS to 22.2 MBq/mL. The resulting solution was inculcated for 10-15 min before centrifugation with the Centrifree Ultrafiltration Devices (Millipore Sigma). Aliquots of the ultrafiltrate (C_{free}) and the plasma mixture (Cₜₒₜₐₗ) were measured for radioactive concentration in a Wallac Wizard 2480 gamma counter. This process was performed in triplicate to determine the plasma free fraction (fₚ) of [¹¹C]13.

Upon PET data acquisition, twenty three arterial blood samples were drawn by sampling every 30 seconds for the first 5 minutes followed by a decreased frequency of every 15 minutes till the end of the scan. The plasma samples were obtained as supernatant of the centrifugated whole-blood samples. The metabolism of [¹¹C]13 was evaluated using selected plasma samples from 5, 10, 15, 30, 60, 90, and 120 minutes. The amount of the intact [¹¹C]13 in plasma samples were measured by the previously described automated column switching radioHPLC system. Briefly, the plasma sample was trapped on a capture column (Waters Oasis HLB 30 μm) with a mobile phase of water: acetonitrile (99:1) at 1.8 mL/min (Waters 515 pump). After 4 minutes, the sample was transferred to an analytical column (Waters XBridge BEH C18, 130 A, 3.5 μm, 4.6 mm×100 mm) by backflushing the catch column with a mobile phase of acetonitrile: 0.1M ammonium formate in water (45:55) at 1 mL/min (Waters 515 pump) with 0.1% of TFA (pH 2.5). The eluent from the analytical column was collected in 1-minute intervals and the radioactivity was measured to determine the parent fraction in plasma (% PP) with a Wallac Wizard 2480 gamma counter. The radioactivity concentration (C(t)) measured from the well counter was expressed as kBq/cc. Therefore, the radioactivity time courses using standardized uptake value (SUV) was calculated as SUV(t)=C(t)/(ID/BW), where ID standards for injected dose in MBq and BW means body weight in kg. The time courses of % PP(t) were fitted with a sum of two decaying exponentials plus a constant. The resulting model fit and the (Cow(t)) in plasma were multiplied to derive the metabolite-corrected arterial input function for kinetic modeling.

Image processing and analyses. All PET data were processed with an in-house developed MATLAB software that uses FSL. The PET images were first co-registered to the structural T1-weighted magnetization-prepared rapid gradient-echo (MEMPRAGE) images, which were aligned into an (magnetic resonance) MR monkey template space. The resulting transformation was then applied to PET images. Regional TACs were extracted from the native PET image space for specific ROIs.

The extracted TACs were modeled via the reversible one- (1T) and two- (2T) tissue compartment model configurations with the metabolite-corrected arterial plasma input function. The 2T model was assessed in its irreversible (k₄=0) and reversible configurations. A fixed vascular contribution of the WB radioactivity to the PET signal was set to 5%. The kinetic parameters were estimated using the nonlinear weighted least-squares fitting and the frame durations were chosen for the weights. Regional total volume of distributions (V_{T}) were calculated from the estimated microparameters following the consensus nomenclature reported by Innis et al. The stability of V_{T} estimates was assessed by progressively truncating the PET data in 10 min increment from the full duration of 120 min to 60 min. Additionally, the Logan graphical analysis technique was also assessed to generate V_{T} estimates with different cutoff time t*.

### Additional Experimental Details

Preparation of mGluR2 homology model for NAMs: The target sequence having 872 residues used for building the model for mGluR2 was listed below:

(SEQ ID NO: 1)
MGSLLALLALLLLWGAVAEGPAKKVLTLEGDLVLGGLFPVHQKGGPAED
CGPVNEHRGIQRLEAMLFALDRINRDPHLLPGVRLGAHILDSCSKDTHA
LEQALDFVRASLSRGADGSRHICPDGSYATHGDAPTAITGVIGGSYSDV
SIQVANLLRLFQIPQISYASTSAKLSDKSRYDYFARTVPPDFFQAKAMA
EILRFFNWTYVSTVASEGDYGETGIEAFELEARARNICVATSEKVGRAM
SRAAFEGVVRALLQKPSARVAVLFTRSEDARELLAASQRLNASFTWVAS
DGWGALESVVAGSEGAAEGAITIELASYPISDFASYFQSLDPWNNSRNP
WFREFWEQRFRCSFRQRDCAAHSLRAVPFEQESKIMFVVNAVYAMAHAL
HNMHRALCPNTTRLCDAMRPVNGRRLYKDFVLNVKFDAPFRPADTHNEV
RFDRFGDGIGRYNIFTYLRAGSGRYRYQKVGYWAEGLTLDTSLIPWASP
SAGPLPASRCSEPCLQNEVKSVQPGEVCCWLCIPCQPYEYRLDEFTCAD
CGLGYWPNASLTGCFELPQEYIRWGDAWAVGPVTIACLGALATLFVLGV
FVRHNATPVVKASGRELCYILLGGVFLCYCMTFIFIAKPSTAVCTLRRL
GLGTAFSVCYSALLTKTNRIARIFGGAREGAQRPRFISPASQVAICLAL
ISGQLLIVVAWLVVEAPGTGKETAPERREVVTLRCNHRDASMLGSLAYN
VLLIALCTLYAFKTRKCPENFNEAKFIGFTMYTTCIIWLAFLPIFYVTS
SDYRVQTTTMCVSVSLSGSVVLGCLFAPKLHIILFQPQKNVVSHRAPTS
RFGSAAARASSSLGQGSGSQFVPTVCNGREVVDSTTSSL

A hybrid model was generated in YASARA from the above sequence and the template structures with the PDB IDs, 4009, 40R2, 6N52 and 5KZN. These structures were obtained after a BLAST search against the PDB of the above mGluR2 sequence. YASARA generated 17 models initially from these structures and finally a hybrid model was generated using the best parts from these 17 initial models, to increase the accuracy beyond each contributor. Figure S1 shows the hybrid model generated in YASARA with initial model in blue and hybridized parts in a different color. The resulting hybrid model obtained the following quality Z-scores (Table S1). Figure S1: The figure shows the initial model in blue, and all hybridized parts in different colors. Table S1: Z-Scores for the hybrid model generated on YASARA.

Structural evaluation of mGluR2 model: This hybrid model was further validated by the following methods. MODFOLD results: The model generated was validated using ModFOLD. The confidence and P-value for this model is HIGH: 1.001 E⁻³ with the global model quality score of 0.4433, indicating it a complete and confident model for mGluR2. The p-value represents the probability of each model being incorrect. The p-value for this model is 0.001001, meaning there is only a 1/1001 chance of this model being incorrect. Figure S2: This image was generated by ModFOLD based on residue accuracy prediction for the model. Blue is for high accuracy through green, yellow, orange to red which is for low accuracy. Structure Analysis Verification Server (SAVES) results: The second server used to validate this model was SAVES and its components, VERIFY 3D and ERRAT. VERIFY 3D scores as a function of sequence number for the model. As Figure S3 shows, VERIFY 3D assigned a 3D-1D score of >0.2 for at least 87.23% of the amino acids. This implies that the model is compatible with its sequence. Figure S3: Verify 3D scores for the hybrid model.

ERRAT scores as a function of sequence number for the hybrid model generated by YASARA. Figure S4, shows good score, regions that can be rejected at 95% confidence and regions that can be rejected at 99% confidence. As Figure S4 shows, the hybrid model contains significantly low 99% rejection regions. The quality factor for this model is 95.9. Therefore, it is a good model according to ERRAT.

SWISS Model-QMEAN results: QMEAN is a composite scoring function which derives both global and local absolute quality estimates based on one single model. The QMEAN score for this hybrid model is −1.43. Shown in Figure S5 is an image showing the sequence of the protein colored by local quality. Figure S5: Image generated by QMEAN showing the local quality of the hybrid model. Better quality regions and lower quality regions are shown. Figure S6: Image showing the local quality of the structure as a function of sequence number, generated by QMEAN.

Ramachandran Plot: The hybrid model generated by YASARA was further evaluated via Ramachandran plot. As Figure S7 shows 89.6% (643) of the residues lie in the favored regions and 10.2% (73) lie in the additionally allowed regions. There are 0.1% (1) residues in the generously allowed regions and no residues in disallowed regions. This is further evidence of a quality model structure. Figure S7: A Ramachandran plot for the hybrid model. Plot generated with the SAVES server.

Prediction of Binding Site: Partial Order Optimum Likelihood (POOL) was used to predict the key binding residues in the allosteric binding site. The identified top residues are listed below and the ones that are reported previously are highlighted in bold. Cys560, Cys606, Leu609, Cys616, Tyr617, Phe623, Arg635, Arg636, Gly638, Leu639, Gly640, Thr641, Phe643, Val645, Cys646, Tyr647, Leu650, Lys653, Cys683, His723, Tyr734, Asn735, Ile739, Cys742, Tyr781, Tyr787, Cys795, Val798, Ser801, Lys813. Figure S8: Position of the allosteric binding site for NAMs.

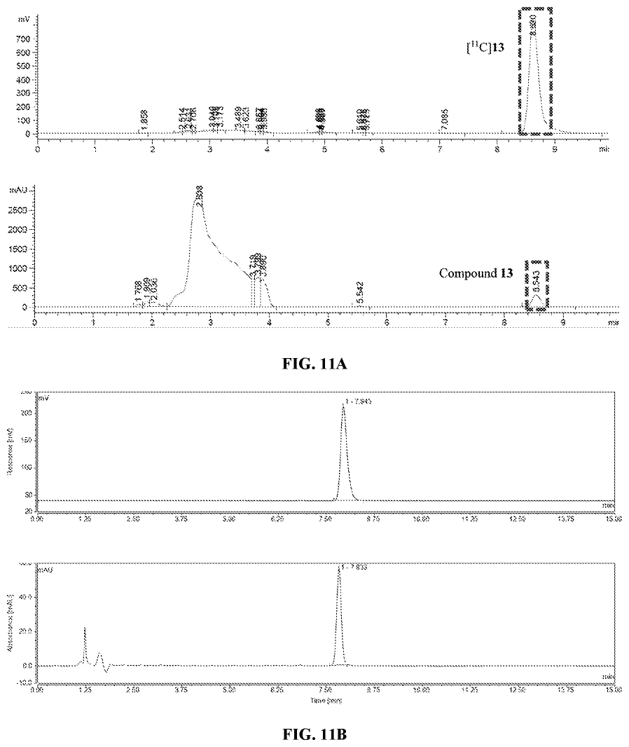

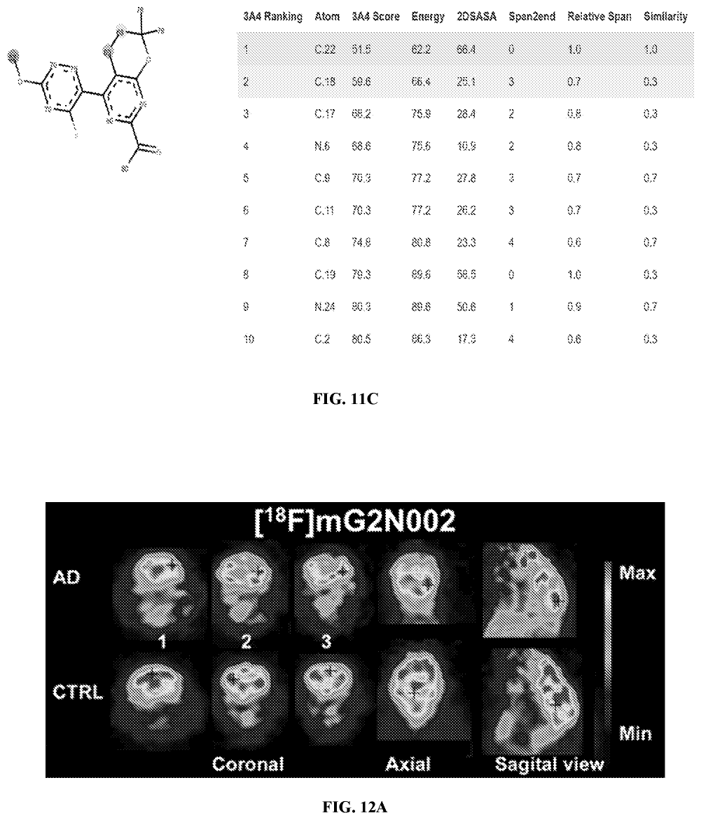

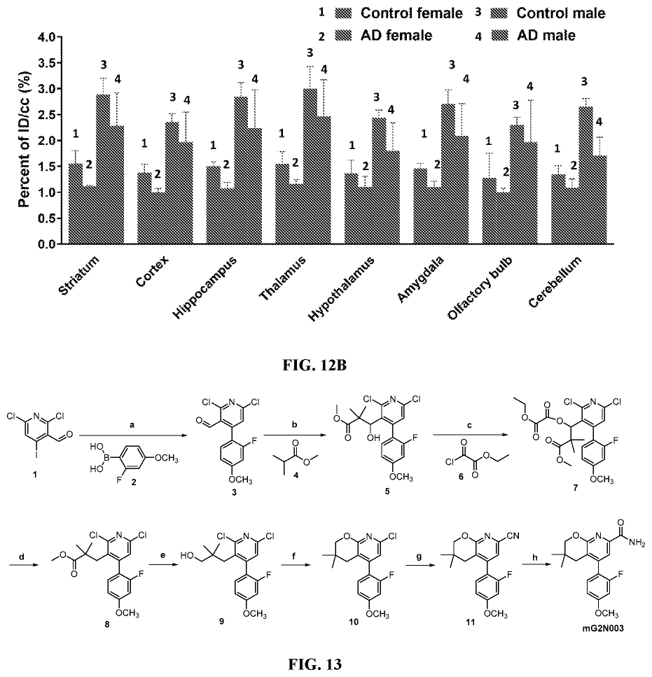

Purification and confirmation of [¹¹C]13: Figure S9 shows the semi-preparative HPLC spectra for the purification of [¹¹C]13 from the reaction mixture. The HPLC radioactivity trace is shown at the top and the UV trace is shown at the bottom. The retention time of [¹¹C]13 was 8.62 min under the following HPLC conditions: Column: Waters XBridge, C18, 250×10 mm, 5μ; Wavelength of 254 nM; Mobile phase: acetonitrile/water/Et₃N (50/50/0.1%) at a flow rate of 5 mL/min. Figure S9. Purification of [¹¹C]13 from reaction mixture via semi-preparative HPLC. The identity of the purified and formulated [¹¹C]13 was confirmed by co-injecting with the unlabeled compound 13 in an analytical HPLC system (Figure S10). The radioHPLC trace of [¹¹C]13 spectrum is shown in top and the UV trace of reference 13 is shown in bottom. The retention time of [¹¹C]13 was 8.11 min under the following HPLC conditions: Column: Waters, XBridge, C18, 4.6×150 mm 3.5μ; Wavelength of 254 nM; Mobile phase: acetonitrile/water/Et₃N (45/55/0.1%) at a flow rate of 1 mL/min. Figure S1. Analytical HPLC Spectra for formulated [¹¹C]13. Prediction of the metabolism sites of 13 with SMARTCyp: results are shown in FIG. 11C.

### Abbreviations Used

CNS, central nervous system; mGluR2, metabotropic glutamate receptor 2; PET, positron emission tomography; PAM, positive allosteric modulator; NAM, negative allosteric modulator; BBB, blood-brain barrier; 7-TM, seven transmembrane; Pgp-BCRP, P-glycoprotein and the breast cancer resistance protein, P-glycoprotein; CHO, chinese hamster ovary; DMF, dimethylformamide; DMA, dimethylacetamide; EOS, end of synthesis; LCMS, Liquid chromatography-mass spectrometry; NHP, non-human primate; ROI, region of interest; TAC, time-activity curve; fp, plasma free fraction; WB, whole-blood; PL, plasma; AIC, Akaike information criteria; NMT, NIMH macaque template; SUV, standardized uptake value; VT, regional total volume of distribution; MEMPRAGE, magnetization-prepared rapid gradient-echo; USP, United States Pharmacopeia; MLEM, maximum-likelihood expectation-maximization.

### BRIEF SUMMARY

Metabotropic glutamate receptor 2 (mGluR2) is a therapeutic target for the treatment of several neuropsychiatric disorders and conditions. The role of mGluR2 function in etiology could be unveiled by in vivo imaging using positron emission tomography (PET). In this regard, 5-(2-fluoro-4-[¹¹C]methoxyphenyl)-2,2-dimethyl-3,4-dihydro-2H-pyrano[2,3-b]pyridine-7-carboxamide ([¹¹C]13), a potent negative allosteric modulator (NAM), was developed to support this endeavor. Radioligand [¹¹C]13 was synthesized via the O-[¹¹C]methylation of phenol 24 with a high molar activity of 212±76 GBq/μmol (n=5) and excellent radiochemical purity (>99%). PET imaging of [¹¹C]13 in rats demonstrated its superior brain heterogeneity, particularly in the regions of striatum, thalamus, hippocampus, and cortex. Accumulation of [¹¹C]13 in these regions of interest (ROIs) was reduced with 5 pretreatment of mGluR2 NAMs, VU6001966 (9) and MNI-137 (26), the extent of which revealed a time-dependent drug effect of the blocking agents. In a nonhuman primate, [¹¹C]13 selectively accumulated in mGluR2-rich regions, especially in different cortical areas, putamen, thalamus, and hippocampus, and resulted in high-contrast brain images. The regional total volume of distribution (V_{T}) estimates of [¹¹C]13 decreased by 14% after the pretreatment with 9. Therefore, [¹¹C]13 is useful, e.g., for translational PET imaging studies of mGluR2 function.

### Example 2—Synthesis and Activity of mG2N003

Synthetic scheme detailing synthesis of compound mG2N003 is shown in 15 FIG. 13. Referring to FIG. 13:

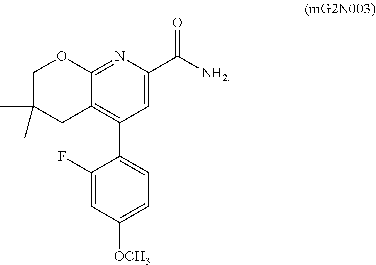 [SMILES: COc1ccc(-c2cc(C(N)=O)nc3c2CC(C)(C)CO3)c(F)c1]

2,6-Dichloro-4-(2-fluoro-4-methoxyphenyl)nicotinaldehyde (3). To a solution of 2,6-dichloro-4-iodonicotinaldehyde (1, 2.0 g, 6.62 mmol) in 1,4-dioxane/water (27 mL/2.7 mL) was added (2-fluoro-4-methoxyphenyl)boronic acid (2, 1.23 g, 7.28 mmol), Pd(dppf)C12 (0.24 g, 0.33 mmol), and Na₂CO₃ (1.4 g, 13.24 mmol). The mixture was stirred at 100° C. for 3 h. The solvent was removed under vacuum and the residue was purified by silica flash column chromatography to give the product as a yellow solid (1.04 g, 52.3% yield). LC-MS [M−H]⁺=297.1.

Methyl 3-(2,6-dichloro-4-(2-fluoro-4-methoxyphenyl)pyridin-3-yl)-3-hydroxy-2,2-dimethylpropanoate (5). To a solution of methyl isobutyrate (0.45 mL, 3.92 mmol) in THF (4.66 mL) under nitrogen at −60° C. was added LDA (4, 0.42 g, 3.92 mmol). The reaction was stirred for 30 min at −60° C. Then, 2,6-dichloro-4-(2-fluoro-4-methoxyphenyl)nicotinaldehyde (3, 0.98 g, 3.26 mmol) was added slowly over 30 min. The reaction was stirred at −60° C. for another 1 hour before its was quenched with saturated ammonium chloride solution (10 mL) and extracted with ethyl acetate (3×10 mL). The combined organic extracts were concentrated under reduced pressure and the residue was purified by silica flash column chromatography to give the product as a colorless oil (1.09 g, 83.2% yield). LC-MS [M+H]⁺=403.3.

1-(2,6-dichloro-4-(2-fluoro-4-methoxyphenyl)pyridin-3-yl)-3-methoxy-2,2-dimethyl-3-oxopropyl ethyl oxalate (7). A mixture of 5 (1.0 g, 2.49 mmol), ethyl 2-chloro-2-oxoacetate (6, 0.56 mL, 4.98 mmol) and pyridine (0.4 mL, 4.98 mmol) in dichloromethane (16 mL) was stirred at 25° C. for 12 h. The reaction mixture was quenched with water (20 mL), extracted with ethyl acetate (3×20 mL). The combined organic extracts were concentrated under reduced pressure and the residue was purified by silica flash column chromatography to give the product as a pale yellow waxy solid (1.14 g, 91.2% yield). LC-MS [M+H]⁺=502.1.

Methyl 3-(2,6-dichloro-4-(2-fluoro-4-methoxyphenyl)pyridin-3-yl)-2,2-dimethylpropanoate (8). To a mixture of 7 (0.98 g, 1.95 mmol) and Bu₃SnH (1.0 M in hexane, 4.0 mL, 4.0 mmol) in toluene (20 mL) was added AIBN (0.2 M in toluene, 3.72 mL, 0.74 mmol). The reaction was stirred at 80° C. for 1 h. The solvent was then removed under reduced pressure. The resulting residue was purified by silica flash column chromatography to give the product as a colorless oil (0.23 g, 30.7% yield). LC-MS [M+H]⁺=386.1.

3-(2,6-Dichloro-4-(2-fluoro-4-methoxyphenyl)pyridin-3-yl)-2,2-dimethylpropan-1-ol (9). To a solution of 8 (0.2 g, 0.52 mmol) in THF (6.2 mL) was added LiBH₄ (0.022 g, 1.04 mmol) under N2 atmosphere. The reaction was stirred at 60° C. for 4 h. Then, it was quenched with saturated ammonium chloride solution (10 mL) and extracted with ethyl acetate (3×10 mL). The combined organic layers were dried over magnesium sulfate and evaporated under vacuum. The resulting residue was purified by silica flash column chromatography to give the product as a colorless oil (0.068 g, 36.6% yield). LC-MS [M+H]⁺=358.1.

7-Chloro-S-(2-fluoro-4-methoxyphenyl)-3,3-dimethyl-3,4-dihydro-2H-pyrano[2,3-b]pyridine (10). A mixture of 9 (48 mg, 0.134 mmol) and Cs₂CO₃ (87.3 mg, 0.268 mmol) in acetonitrile (2.0 mL) was stirred at 70° C. for 12 h. The solvent was then removed under reduced pressure and the residue was purified by silica flash column chromatography to give the product as a colorless oil (36 mg, 83.5% yield). LC-MS [M+H]⁺=322.1.

5-(2-Fluoro-4-methoxyphenyl)-3,3-dimethyl-3,4-dihydro-2H-pyrano[2,3-b]pyridine-7-carbonitrile (11). To a solution of 10 (10.0 mg, 0.031 mmol) in dimethylformamide (1.2 mL) was added zinc cyanide (12.0 mg, 0.102 mmol) and tetrakis(triphenylphosphine)palladium(0) (10.0 mg, 0.0086 mmol) in a microwave tube. The mixture was heated to 160° C. in a microwave synthesizer (CEM, Discover SP) for 30 min. The reaction was cooled to room temperature, washed with water (20 mL) and extracted with ethyl acetate (3×20 mL). The combined organic layers were dried over anhydrous MgSO₄. The solvent was removed under reduced pressure and the residue was purified by silica flash column chromatography to give the product as a pale-yellow oil (5.0 mg, 51.6% yield). LC-MS [M+H]⁺=313.2.

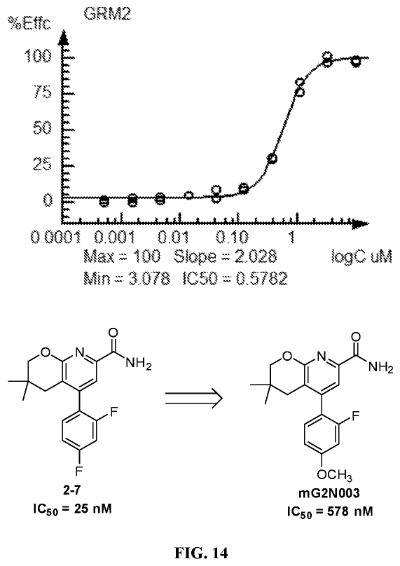

5-(2-Fluoro-4-methoxyphenyl)-3,3-dimethyl-3,4-dihydro-2H-pyrano[2,3-b]pyridine-7-carboxamide (mG2N003). To a solution of 11 (10.0 mg, 0.032 mmol) in acetone (1.1 mL) was added a solution of sodium percarbonate (24.3 mg, 0.155 mmol) in water (0.6 mL) dropwise. The mixture was stirred at room temperature for 1h. After the reaction was completed, the mixture was diluted with water (5 mL) and extracted with ethyl acetate (3×5 mL). The combined organic layers were dried over anhydrous MgSO₄. The solvent was removed under reduced pressure and the residue was purified by silica flash column chromatography to give the product as a pale-yellow solid (8.0 mg, 75.7% yield). LC-MS [M+H]⁺=331.3. mG2N003 Binding Potency was determined according to protocol A, Yuan, G. et al. J. Med. Chem. 2022, 65(3), 2593-2609. See FIG. 14. Replacing p-F atom in compound 2-7 with a methoxy group leads to >20× decrease in potency toward GRM2. IC₅₀=578 nM (IC₅₀=25 nM for compound 2-7).

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A compound of Formula (I): 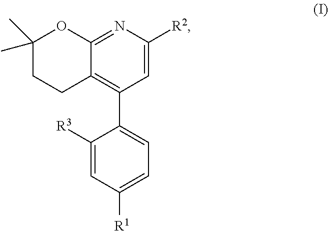
or a pharmaceutically acceptable salt thereof, wherein:
R¹, R², and R³ are each independently selected from halo, CN, C(═O)NH₂, C₁₋₃ alkyl, C₁₋₃ haloalkyl, C₁₋₃ alkoxy, and C₁₋₃ haloalkoxy; and
one of R¹, R², and R³ comprises a radioisotope selected from ¹¹C and ¹⁸F.

2. The compound of claim 1, wherein R¹ comprises a radioisotope selected from ¹¹C and ¹⁸F.

3. The compound of claim 2, wherein R¹ comprises ¹¹C.

4. The compound of claim 2, wherein R¹ comprises ¹⁸F.

5. The compound of claim 2, wherein R¹ is selected from ¹⁸F, ¹¹CN, ¹¹C(═O)NH₂, H₃¹¹C—, ¹⁸FCH₂CH₂—, ¹¹CH₃O—, ¹⁸FCH₂CH₂O—, ¹⁸FCH₂CH₂CH₂O—, ¹⁸FCD₂O—, and ¹⁸FCH₂O—.

6. The compound of claim 2, wherein R¹ is selected from ¹⁸F, ¹¹CN, ¹¹CH₃O—, ¹⁸FCH₂CH₂O—, ¹⁸FCH₂CH₂CH₂O—, ¹⁸FCD₂O—, and ¹⁸FCH₂O—.

7. The compound of claim 6, wherein R¹ is selected from ¹⁸F and ¹¹CH₃O—.

8. The compound of claim 7, having formula: 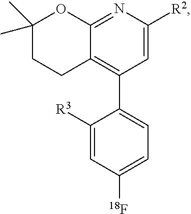
or a pharmaceutically acceptable salt thereof.

9. The compound of claim 7, having formula: 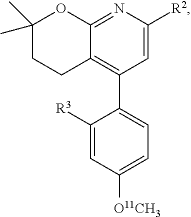
or a pharmaceutically acceptable salt thereof.

10. The compound of any one of claims 1-9, wherein:
R² is selected from halo, CN, and C(═O)NH₂; and
R³ is selected from halo, C₁₋₃ alkoxy, and C₁₋₃ haloalkoxy.

11. The compound of claim 10, wherein:
R² is selected from CN and C(═O)NH₂; and
R³ is selected from halo and C₁₋₃ alkoxy.

12. The compound of any one of claims 1-11, wherein R³ is halo.

13. The compound of claim 1, wherein the compound of Formula (I) is selected from any one of the following compounds: 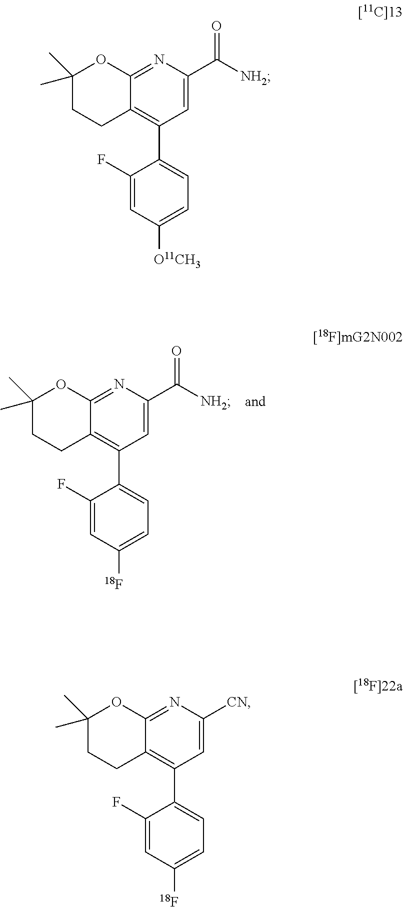
or a pharmaceutically acceptable salt thereof.

14. The compound of claim 13, having formula: 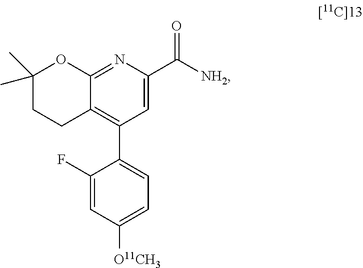
or a pharmaceutically acceptable salt thereof.

15. The compound of claim 13, having formula 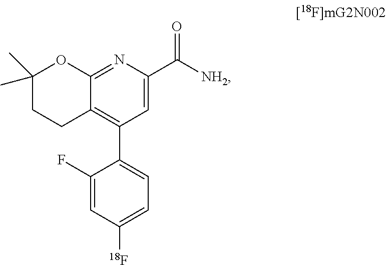 [SMILES: CC1(C)CCc2c(-c3ccc([18F])cc3F)cc(C(N)=O)nc2O1]
or a pharmaceutically acceptable salt thereof.

16. The compound of claim 1, wherein the compound of Formula (I) is selected from any one of the following compounds: 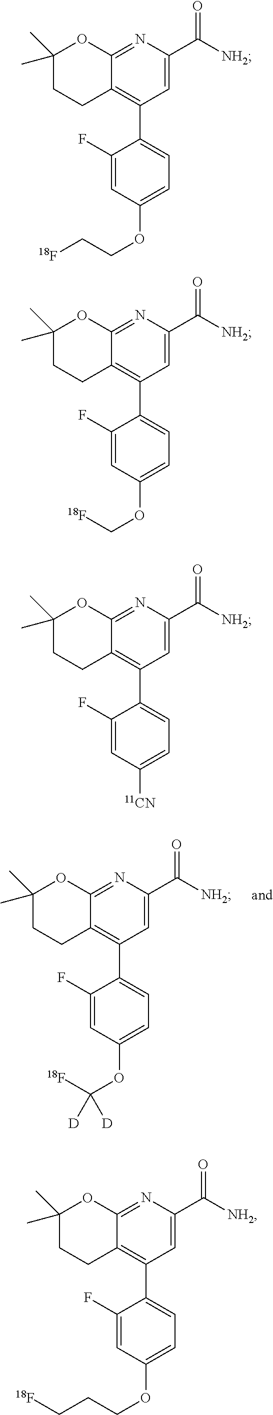
or a pharmaceutically acceptable salt thereof.

17. The compound of claim 1, wherein R² comprises a radioisotope selected from ¹¹C and ¹⁸F.

18. The compound of claim 17, wherein R² comprises ¹¹C.

19. The compound of claim 17, wherein R² comprises ¹⁸F.

20. The compound of claim 17, wherein R² is selected from ¹⁸F, ¹¹CN, ¹¹C(═O)NH₂, H³₁₁C—, ¹⁸FCH₂CH₂—, ¹¹CH₃O—, ¹⁸FCH₂CH₂O—, ¹⁸FCH₂CH₂CH₂O—, ¹⁸FCD₂O—, and ¹⁸FCH₂O—.

21. The compound of claim 20, wherein R¹ is selected from ¹¹CN and ¹¹C(═O)NH₂.

22. The compound of claim 21, having formula: 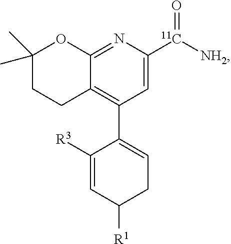
or a pharmaceutically acceptable salt thereof.

23. The compound of any one of claims 17-22, wherein R² and R³ are each independently selected from halo, C₁₋₃ alkoxy, and C₁₋₃ haloalkoxy.

24. The compound of claim 23, wherein R² and R³ are each independently selected from halo and C₁₋₃ alkoxy.

25. The compound of claim 23, wherein R² and R³ are each independently halo.

26. The compound of claim 1, wherein the compound of Formula (I) is selected from any one of the following compounds: 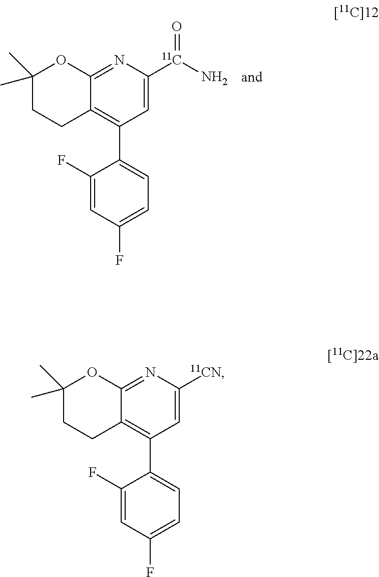
or a pharmaceutically acceptable salt thereof.

27. The compound of claim 26, having formula: 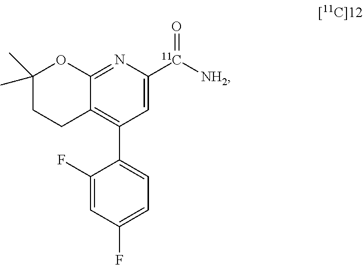 [SMILES: CC1(C)CCc2c(-c3ccc(F)cc3F)cc([11C](N)=O)nc2O1]
or a pharmaceutically acceptable salt thereof.

28. The compound of claim 1, wherein R³ comprises a radioisotope selected from ¹¹C and ¹⁸F.

29. The compound of claim 28, wherein R³ comprises ¹¹C.

30. The compound of claim 28, wherein R³ comprises ¹⁸F.

31. The compound of claim 28, wherein R³ is selected from ¹⁸F, ¹¹CN, ¹¹C(═O)NH₂, H³₁₁C—, ¹⁸FCH₂CH₂—, ¹¹CH₃O—, ¹⁸FCH₂CH₂O—, ¹⁸FCH₂CH₂CH₂O—, ¹⁸FCD₂O—, and ¹⁸FCH₂O—.

32. The compound of claim 28, wherein R³ is selected from ¹⁸F, ¹¹CH₃O—, ¹⁸FCH₂CH₂O—, ¹⁸FCH₂CH₂CH₂O—, ¹⁸FCD₂O—, and ¹⁸FCH₂O—.

33. The compound of any one of claims 28-32, wherein:
R¹ is selected from halo, C₁₋₃ alkoxy, and C₁₋₃ haloalkoxy; and
R² is selected from halo, CN, and C(═O)NH₂.

34. The compound of claim 33, wherein:
R¹ is selected from halo and C₁₋₃ alkoxy; and
R² is selected from CN and C(═O)NH₂.

35. The compound of claim 34, wherein R¹ is halo.

36. The compound of claim 1, wherein the compound of Formula (I) is selected from any one of the following compounds: 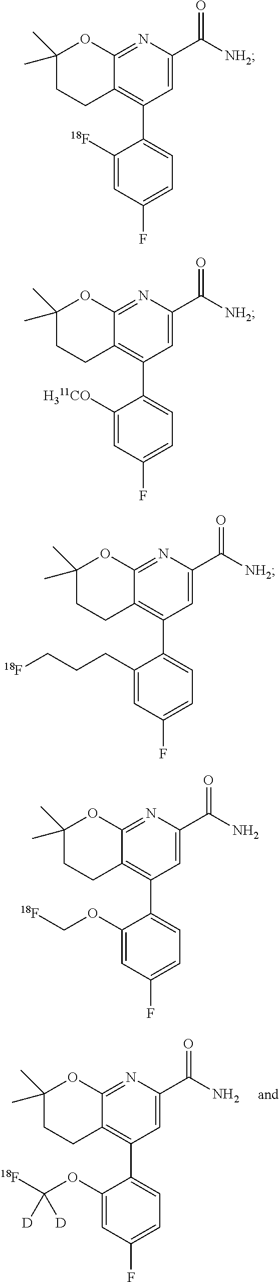 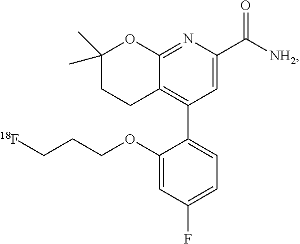 [SMILES: CC1(C)CCc2c(-c3ccc(F)cc3OCCC[18F])cc(C(N)=O)nc2O1]
or a pharmaceutically acceptable salt thereof.

37. A pharmaceutical composition comprising a compound of any one of claims 1-36, or a pharmaceutically acceptable salt thereof, and a pharmaceutically acceptable carrier.

38. A method of imaging a brain of a subject, the method comprising:
i) administering to the subject an effective amount of a compound of any one of claims 1-36, or a pharmaceutically acceptable salt thereof, or a pharmaceutical composition of claim 37;
ii) waiting a time sufficient to allow the compound to accumulate in the brain to be imaged; and
iii) imaging the brain with an imaging technique.

39. The method of claim 38, wherein the compound selectively binds to mGluR2 in the brain.

40. The method of claim 38, wherein imaging the brain comprises imaging striatum, thalamus, hypothalamus, hippocampus, cerebellum, cortex, and/or putamen.

41. The method of any one of claims 38-40, wherein imaging the brain comprises diagnosing the subject with a psychiatric or a neurological disorder associated with mGluR2.

42. A method of monitoring treatment of a psychiatric or a neurological disorder associated with mGluR2 in a subject, the method comprising:
i) administering to the subject an effective amount of a compound of any one of claims 1-36, or a pharmaceutically acceptable salt thereof, or a pharmaceutical composition of claim 37;
ii) waiting a time sufficient to allow the compound of any one of claims 1-36 administered in step i) to accumulate in a brain of the subject;
iii) imaging the brain of the subject with an imaging technique;
iv) administering to the subject a therapeutic agent in an effective amount to treat the psychiatric or the neurological disorder;
v) after iv), administering to the subject an effective amount of a compound of any one of claims 1-36, or a pharmaceutically acceptable salt thereof, or a pharmaceutical composition of claim 37;
vi) waiting a time sufficient to allow the compound of any one of claims 1-36 administered in step v) to accumulate in the brain of the subject;
vii) imaging the brain of the subject with an imaging technique; and
viii) comparing the image of step iii) and the image of step vii).

43. The method of any one of claims 38-42, wherein the imaging technique is selected from positron emission tomography (PET) imaging, positron emission tomography with computer tomography (PET/CT) imaging, and positron emission tomography with magnetic resonance (PET/MRI) imaging.

44. The method of claim 42, wherein the neurological disorder associated with mGluR2 is selected from Alzheimer's disease, Parkinson's disease, dyskinesia, Lewy body disease, Prion disease, motor neuron disease (MND), and Huntington's disease.

45. The method of claim 42, wherein the psychiatric disorder associated with mGluR2 is selected from schizophrenia, psychosis, anxiety, depression, drug abuse, pain, smoking cessation, and epilepsy.


SEQUENCE LISTING (1 sequence)

SEQ ID NO: 1 - protein, 872 aa, Artificial Sequence [sequence omitted]